(19) United States
(12) Patent Application Publication
(10) Pub. No.: US 2004/0167320 A1
(43) Pub. Date: Aug. 26, 2004

(54) Methods of tangential flow filtration and an apparatus therefore

(76) Inventors: Daniel E. Couto, Chelsea, MA (US); Amy Laverdiere, San Francisco, CA (US)

Correspondence Address: GTC BIOTHERAPEUTICS, INC., 175 CROSSING BOULEVARD, SUITE 410, FRAMINGHAM, MA, 01702, US

(21) Appl. No.: 10/635,117

(22) Filed: Aug. 6, 2003

Related U.S. Application Data
(60) Provisional application No. 60/449,786, filed on Feb. 24, 2003.

(51) Int. Cl. C07K 1/34

(52) U.S. Cl. 530/412

(57) ABSTRACT

Processes and apparati are provided for separating molecules of interest from a mixture containing them which comprises subjecting the mixture to an improved method of tangential flow filtration (TFF). The improved TFF was used to clarify, and process various feedstreams for the removal of a molecule of interest. According to a preferred embodiment, a transgenic milk feedstream is stabilized and particulate matter such as fat, casein miscelles and bacteria are removed. The method of TFF used in the current invention utilizes optimized process parameters that include temperature, transmembrane pressure, cross-flow velocity, and milk concentration. Cleaning and storage procedures were also developed to ensure long membrane life. An aseptic filtration step was also developed to remove any bacteria remaining in a clarified transgenic milk feedstream.

### FIELD OF THE INVENTION

The present invention provides an improved method and system of purifying specific target molecules from contaminants. More specifically the methods of the current invention provide for the processing of a sample solution through an improved method of tangential flow filtration that enhances the clarification, concentration and fractionation of a desired molecule from a given feedstream.

### BACKGROUND OF THE INVENTION

The present invention is directed to an improved method of filtration of molecules of interest from a given feedstream. It should be noted that the production of large quantities of relatively pure, biologically active molecules is important economically for the manufacture of human and animal pharmaceutical formulations, proteins, enzymes, antibodies and other specialty chemicals. For production of many polypeptides, antibodies and proteins, recombinant DNA techniques have become the method of choice because large quantities of exogenous proteins or antibodies can be expressed in bacteria, yeast, insect or mammalian cell cultures. More recently, transgenic animals, typically mammals, but also avians or even transgenic plants have been engineered or otherwise modified to produce exogenous proteins, antibodies, or fragments or fusions thereof, in large quantities. The expression of proteins by recombinant DNA techniques for the production of cells or cell parts that function as biocatalysts is also an important application.

Producing recombinant protein involves transfecting host cells with DNA encoding the protein and growing the host cells, transgenic animals or plants under conditions favoring expression of the recombinant protein or other molecule of interest. The prokaryote E. coli has been a favored host system because it can be made to produce recombinant proteins in high yields. However, numerous U.S. patents on the general expression of DNA encoding proteins exist, for a variety of expression platforms from E. coli to cattle have been developed.

With improvements in the production of exogenous proteins or other molecules of interest from biological systems there has been increasing pressure on industry to develop new techniques to enhance and make more efficient the purification and recovery processes for the biologics and pharmaceuticals so produced. That is, with an increased pipeline of new products, there is substantial interest in devising methods to bring these therapeutics, in commercial volumes, to market quickly. At the same time the industry is facing new challenges in terms of developing novel processes for the recovery of transgenic proteins and antibodies from various bodily fluids including milk and urine. The larger the scale of production the more complex these problems often become. In addition, there are further challenges imposed in terms of meeting product purity and safety, notably in terms of virus safety and residual contaminants, such as DNA and host cell proteins that might be required to be met by the various governmental agencies that oversee the production of biologically useful pharmaceuticals.

Several methods are currently available to separate molecules of biological interest, such as proteins, from mixtures thereof. One important such technique is affinity chromatography, which separates molecules on the basis of specific and selective binding of the desired molecules to an affinity matrix or gel, while the undesirable molecule remains unbound and can then be moved out of the system. Affinity gels typically consist of a ligand-binding moiety immobilized on a gel support. For example, GB 2,178,742 utilizes an affinity chromatography method to purify hemoglobin and its chemically modified derivatives based on the fact that native hemoglobin binds specifically to a specific family of poly-anionic moieties. For capture these moieties are immobilized on the gel itself. In this process, unmodified hemoglobin is retained by the affinity gel, while modified hemoglobin, which cannot bind to the gel because its poly-anion binding site is covalently occupied by the modifying agent, is removed from the system. Affinity chromatography columns are highly specific and thus yield very pure products; however, affinity chromatography is a relatively expensive process and therefore very difficult to put in place for commercial operations.

Typically, genetically engineered biopharmaceuticals are purified from a supernatant containing a variety of diverse host cell contaminants. Reversed-phase high-performance liquid chromatography (RP-HPLC) can be used for protein purification because it can efficiently separate molecular species that are exceptionally similar to one another in terms of structure or weight. Procedures utilizing RP-HPLC have been published for many molecules. McDonald and Bidlingmeyer, “Strategies for Successful Preparative Liquid Chromatography”, PREPARATIVE LIQUID CHROMATOGRAPHY, Brian A. Bidlingmeyer (New York: Elsevier Science Publishing, 1987), vol. 38, pp. 1-104; Lee et al., Preparative HPLC. 8th Biotechnology Symposium, Pt. 1, 593-610 (1988).

The use of membranes in the recovery processes for molecular products at industrial scale, and the associated use of many types of membranes and membrane techniques is also known. In these methods the essential feature is that particles, suspended in a liquid feedstream are separated on the basis of their size. In the simplest form of this process, a solution is forced under pressure through a filter membrane with pores of a defined size. Particles larger than the pore size of the membrane filter are retained, while smaller solutes are carried convectively through the membrane with the solvent. Such membrane filtration processes generally falls within the categories of reverse osmosis, ultrafiltration, and microfiltration, depending on the pore size of the membrane.

It is also important to mention that membrane filtration as a separation technique is widely used in the biotechnology field. Depending on membrane type, it can be classified as microfiltration or ultrafiltration. Microfiltration membranes, with a pore size between 0.1 and 10 μm, are typically used for clarification, sterilization, removal of microparticulates, or for cell harvests. Ultrafiltration membranes, with much smaller pore sizes between 0.001 and 0.1 μm, are used for separating out and concentrating dissolved molecules (protein, peptides, nucleic acids, carbohydrates, and other biomolecules), for exchange buffers, and for gross fractionation.

Currently, there are two main membrane filtration methods: Single Pass or Direct Flow Filtration (DFF) and Crossflow or Tangential Flow Filtration (TFF). With regard to TFF, it is an ultrafiltration system that has been designed to control the fluid flow pattern of a feedstream so as to enhance transport of the retained solute away from the membrane surface and back into the bulk of the feed. In this process the feedstream is re-circulated at high velocities at a vector tangential to the plane of the membrane. This is done to increase the mass-transfer co-efficient to allow for back diffusion. The fluid flowing in a direction parallel to the filter membrane also acts to clean the filter surface continuously and thereby prevents clogging.

However, limitations exist on the degree of protein purification achievable in ultrafiltration. These limits are due mainly to the phenomena of concentration polarization, fouling, and the wide distribution in the pore size of most membranes. Therefore solute discrimination is often poor. See, e.g., Porter, ed., HANDBOOK OF INDUSTRIAL MEMBRANE TECHNOLOGY (Noyes Publications, Park Ridge, N.J., 1990), pp. 164-173.

A polarized layer of solutes acts as an additional filter and essentially acts in series with the original ultra-filter. This action provides significant resistance to the filtration of a given solvent. The degree of polarization increases with increasing concentration of retained solute in the feed, and can lead to a number of seemingly anomalous or unpredictable effects in real systems. For example, under highly polarized conditions, filtration rates may increase only slightly with increasing pressure, in contrast to unpolarized conditions, where filtration rates are usually linear with pressure. Use of a more open, higher-flux membrane may not increase the filtration rate, because the polarized layer is providing the limiting resistance to filtration. The situation is further complicated by interactions between retained and eluted solutes.

A result of concentration polarization and fouling processes is the inability to make effective use of the macromolecular fractionation capabilities of ultrafiltration membranes for the large-scale resolution of macromolecular mixtures such as blood plasma proteins. See Michaels, “Fifteen Years of Ultrafiltration: Problems and Future Promises of an Adolescent Technology”, in ULTRAFILTRATION MEMBRANES AND APPLICATIONS, POLYMER SCIENCE AND TECHNOLOGY, 13 (Plenum Press, N.Y., 1979, Anthony R. Cooper, ed.,), pp. 1-19.

Consequently, the use of other and additional techniques for the separation of a wider variety of biomolecules is difficulty. That is, the use of membrane ultrafiltration for large-scale complex macromolecular mixture-separations performed by such techniques as gel permeation, adsorption, or ion-exchange chromatography, selective precipitation, or electrophoresis is exceptionally difficult, and not useful in commercial applications. TFF solves this clogging problem by re-circulating the mixture.

The use of tangential flow filtration for the separation of materials is known. Marinaccio et al., U.S. Pat. No. 4,888,115 discloses the process (termed “cross-flow”) for use in the separation of biological liquids such as blood components for plasmapheresis. In this process, blood is passed tangentially to (i.e., across) an organic polymeric microporous filter membrane, and particulate matter is removed. In another example of current art, tangential flow filtration has been disclosed for the filtration of beer solutions (Shackleton, EP 0,208,450, published Jan. 14, 1987) specifically for the removal of particulates such as yeast cells and other suspended solids. Kothe et al., (U.S. Pat. No. 4,644,056, issued Feb. 17, 1987) disclose the use of this process in the purification of immunoglobulins from milk or colostrum, and Castino (U.S. Pat. No. 4,420,398, issued Dec. 13, 1983) describes its use in the separation of antiviral substances such as interferons from broths containing these substances as well as viral particles and the remains of cell cultures from which they are derived.

Tangential flow filtration units have been employed in the separation of bacterial enzymes from cell debris (Quirk et al., 1984, Enzyme Microb. Technol., 6(5):201). Using this technique, Quirk et al. were able to isolate enzyme in higher yields and in less time than using the conventional technique of centrifugation. The use of tangential flow filtration for several applications in the pharmaceutical field has been reviewed by Genovesi (1983, J. Parenter. Aci. Technol., 37(3):81), including the filtration of sterile water for injection, clarification of a solvent system, and filtration of enzymes from broths and bacterial cultures.

However, the precise control of particle size needed for commercial applications of the technology is difficult and generally has not been successful. In the present invention the use of tangential flow filtration has been adapted to separate particles according to size in a commercially efficient and important process. The use of filters of selected sizes, and further, the sequential use or serial attachment of filters of different sizes (i.e., a filtering system) is disclosed for the separation of particles to obtain particles of a specifically desired size range.

There is also a need in the art for an efficient protocol for selectively separating molecules such as peptides, polypeptides, and non-peptidyl compounds from other molecules using a process that increases yield, is less expensive and is less denaturing. In particular, there is a need for purification techniques to allow the separation of a molecule of interest from a fermentation broth as utilized in cell culture or a milk feedstream produced by a transgenic mammal.

One such molecule of interest that can be purified from a cell culture broth or a transgenic milk feedstream is human albumin. Human albumin was the first natural colloid composition for clinical use as a blood volume expander, and it is the standard colloidal agent for comparison with other colloidal molecules. Other molecules of interest include without limitation, human alpha-fetoprotein, antibodies, Fc fragments of antibodies and fusion molecules wherein a human albumin or alpha-fetoprotein protein fragment acts as the carrier molecule.

The methods of the current invention also provide precise combinations of filters and conditions that allow the optimization of the yield of molecules of interest from a given feedstream. In these methods important the process parameters such as pH and temperature are precisely manipulated.

It is an object of the present invention to provide tangential-flow filtration processes for separating species such as particles and molecules by size, which processes are selective for the species of interest, resulting in higher-fold purification thereof.

It is another object to provide improved filtration processes, including ultrafiltration processes, for separating biological macromolecules such as proteins which processes minimize concentration polarization and do not increase flux.

It is another object to provide a filtration process that can separate by size species that are less than ten-fold different in size and do not require dilution of the mixture prior to filtration.

These and other objects will become apparent to those skilled in the art. Other features and advantages of this invention will become apparent in the following detailed description of preferred embodiments of this invention, taken with reference to the accompanying drawings

The biologics industry is becoming increasingly concerned with product safety and purity as well as cost of goods. The use of tangential flow filtration (TFF), according to the current invention, is a rapid and more efficient method for biomolecule separation. It can be applied to a wide range of biological fields such as immunology, protein chemistry, molecular biology, biochemistry, and microbiology.

### BRIEF DESCRIPTION OF THE DRAWINGS

FIG. 1 Shows a process flow diagram for flow of material from feedstream through TFF to fill and finish.

FIG. 2A Shows the process and equipment set-up for microfiltration.

FIG. 2B Shows the process and equipment set-up for TFF.

FIG. 3 Shows the comparative removal of casein products at high and low temperatures.

FIG. 4 Shows a filtration process flow diagram.

FIG. 5 Shows the transgenics development process from a DNA construct to the production of clarified milk containing a recombinant protein of interest.

FIG. 6 Shows a process equipment schematic for the methods of the current invention.

FIG. 7 Shows the Fluid Dynamic Characteristics of hMAb #A passage through a MF membrane with respect to Crossflow Velocity at Varying TMP. A progressing development is noticed at TMP increases from 12 psi to 20 psi.

FIG. 8 Shows the temperature dependence of a human MAb #A passage through a MF membrane. Both cell culture antibody and Tg antibody are provided.

FIG. 9 Shows SDS PAGE analysis of various fractions from the GTC Microfiltration process. Including a reduction of casein in 4× Clarified milk (Lane #7) compared to whole milk (Lane #3).

FIG. 10 Shows the TFF process, mass balance as well as overall yield of the process according to the invention.

### SUMMARY OF THE INVENTION

Briefly stated, the current invention provides a method for the accelerated processing of human therapeutic proteins, protein fragments, or antibodies from a variety of feedstreams, preferably from transgenic mammalian milk. Therefore, in a preferred embodiment of the current invention the filtration technology developed and provided herein provides a process to clarify, concentrate and fractionate the desired recombinant protein or other molecule of interest from the native components of milk or contaminants thereof. The resulting clarified bulk intermediate is a suitable feed material for traditional purification techniques such as chromatography which are used down stream from the TFF process to bring the product to it's final formulation and purity.

A preferred procotol of the current invention employs three filtration unit operations that clarify, concentrate, and fractionate the product from a given transgenic milk volume containing a molecule of interest. The clarification step removes larger particulate matter, such as fat globules and casein micelles from the product. The concentration and fractionation steps thereafter remove most small molecules, including lactose, minerals and water, to increase the purity and reduce the volume of the resulting product composition. The product of the TFF process is tailor concentrated to a level suitable for optimal down stream purification and overall product stability. This concentrated product is then aseptically filtered to assure minimal bioburden and enhance stability of the product for extended periods of time. The bulk product will realize a purity between 65% and 85% and may contain components such as goat antibodies, whey proteins (β Lactoglobulin, α Lactalbumin, and BSA), and low levels of residual fat and casein. This partially purified product is an ideal starting feed material for conventional down stream chromatographic techniques.

Typical of the products that the current invention can be used to process are immunoglobulin molecules, including without limitation: IgG1 (ex: antibodies directed against arthritis—“Remicade antibody”), IgG4, IgM, IgA, Fc portions, fusion molecules containing a peptide or polypeptide joined to a immunoglobulin fragment. Other proteins that can be processed by the current invention include recombinant proteins, endogenous proteins, fusion proteins, or biologically inactive proteins that can be later processed to restore biological function. Included among these processes, without limitation, are the proteins antithrombin III, human serum albumin, decorin, human alpha fetoprotein urokinase, and prolactin.

### DESCRIPTION OF THE PREFERRED EMBODIMENT

The following abbreviations have designated meanings in the specification:

Abbreviation Key:

BSA Bovine Serum Albumin

CHO Chinese Hamster Ovary cells

CV Crossflow Velocity

DFF Direct Flow Filtration

DV Diafiltration Volume

IEF Isoelectric Focusing

GMH Mass Flux (grams/m²/hour)—also J_{M}

LMH Liquid Flux (liters/m²/hour)—also J_{L}

LPM Liters Per Minute

M Molar

MF Microfiltration

NMWCO Nominal Molecular Weight Cut Off

NWP Normalized Water Permeability

PES Poly(ether)-sulfone

pH A term used to describe the hydrogen-ion activity of a chemical or compound according to well-known scientific parameters.

PPM Parts Per Million

SDS-PAGE SDS (sodium dodecyl sulfate) Poly-Acrylamide Gel electrophoresis

SEC Size Exclusion Chromatography

TFF Tangential Flow Filtration

PEG Polyethylene glycol

TMP Transmembrane Pressure

UF Ultrafiltration

Explanation of Terms:

Clarification

The removal of particulate matter from a solution so that the solution is able to pass through a 0.2 μm membrane.

Colloids

Refers to large molecules that do not pass readily across capillary walls. These compounds exert an oncotic (i.e., they attract fluid) load and are usually administered to restore intravascular volume and improve tissue perfusion.

Concentration

The removal of water and small molecules with a membrane such that the ratio of retained molecules to small molecules increases.

Concentration Polarization

The accumulation of the retained molecules (gel layer) on the surface of the membrane caused by a combination of factors: transmembrane pressure, crossflow velocity, sample viscosity, and solute concentration.

Crossflow Velocity

Velocity of the fluid across the top of the membrane surface. CF=Pi−Po where Pi is pressure at the inlet and Po is pressure at the outlet and is related to the retentate flow rate.

Diafiltration

The fractionation process of washing smaller molecules through a membrane, leaving the larger molecule of interest in the retentate. It is a convenient and efficient technique for removing or exchanging salts, removing detergents, separating free from bound molecules, removing low molecular weight materials, or rapidly changing the ionic or pH environment. The process typically employs a a microfiltration membrane that is employed to remove a product of interest from a slurry while maintaining the slurry concentration as a constant.

Feedstream

The raw material or raw solution provided for a process or method and containing a protein of interest and which may also contain various contaminants including microorganisms, viruses and cell fragments.

Filtrate Flux (J)

The rate at which a portion of the sample has passed through the membrane.

Flow Velocity (V)

The speed at which the fluid passes the surface of the membrane is considered the fluid flow velocity. Product flux will be measured as flow velocity is varied. The relationship between the two variables will allow us to determine an optimal operational window for the flow.

Fractionation

The preferential separation of molecules based on a physical or chemical moiety.

Gel Layer

The microscopically thin layer of molecules that can form on the top of a membrane. It can affect retention of molecules by clogging the membrane surface and thereby reduce the filtrate flow.

Nominal Molecular Weight Cut Off (NMWCO)

The size (kilodaltons) designation for the ultrafiltration membranes. The MWCO is defined as the molecular weight of the globular protein that is 90% retained by the membrane.

Normalized Water Permeability (NWP)

The water filtrate flow rate established at a specific recirculation rate during TFF device initial cleaning. This value is used to calculate membrane recovery.

Molecule of Interest

Particles or other species of molecule that are to be separated from a solution or suspension in a fluid, e.g., a liquid. The particles or molecules of interest are separated from the fluid and, in most instances, from other particles or molecules in the fluid. The size of the molecule of interest to be separated will determine the pore size of the membrane to be utilized. Preferably, the molecules of interest are of biological or biochemical origin or produced by transgenic or in vitro processes and include proteins, peptides, polypeptides, antibodies or antibody fragments. Examples of preferred feedstream origins include mammalian milk, mammalian cell culture and microorganism cell culture such as bacteria, fungi, and yeast. It should also be noted that species to be filtered out include non-desirable polypeptides, proteins, cellular components, DNA, colloids, mycoplasm, endotoxins, viruses, carbohydrates, and other molecules of biological interest, whether glycosylated or not.

Tangential Flow Filtration

A process in which the fluid mixture containing the components to be separated by filtration is re-circulated at high velocities tangential to the plane of the membrane to increase the mass-transfer coefficient for back diffusion. In such filtrations a pressure differential is applied along the length of the membrane to cause the fluid and filterable solutes to flow through the filter. This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

Transmembrane Pressure

The pressure differential gradient that is applied along the length of a filtration membrane to cause fluid and filterable solutes to flow through the filter. In tangential flow systems, highest TMP's are at the inlet (beginning of flow channel) and lowest at the outlet (end of the flow channel). TMP is calculated as an average pressure of the inlet, outlet, and filtrate ports.

Recovery

The amount of a molecule of interest that can be retrieved after processing. Usually expressed as a percentage of starting material or yield.

Retentate

The portion of the sample that does not pass through the membrane, also known as the concentrate. Retentate is being re-circulated during the TFF.

Principles of Tangential Flow Filtration

There are two important variables involved in all tangential flow devices: the transmembrane pressure (TMP) and the crossflow velocity (CF). The transmembrane pressure (TMP) is the force that actually pushes molecules through the pores of the filter. The crossflow velocity is the flow rate of the solution across the membrane. It provides the force that sweeps away larger molecules that can clog the membrane thereby reducing the effectiveness of the process. In practice a fluid feedstream is pumped from the sample feed container source across the membrane surface (crossflow) in the filter and back into the sample feed container as the retentate. Backpressure applied to the retentate tube by a clamp creates a transmembrane pressure which drives molecules smaller than the membrane pores through the filter and into the filtrate (or permeate) fraction. The crossflow sweeps larger molecules, which are retained on the surface of the membrane, back to the feed as retentate. The primary objective for the successful implementation of a TFF protocol is to optimize the TMP and CF so that the largest volume of sample can be filtered without creating a membrane-clogging gel. A TMP is “substantially constant” if the TMP does not increase or decrease along the length of the membrane generally by more than about 10 psi of the average TMP, and preferably by more than about 5 psi. As to the level of the TMP throughout the filtration, the TMP is held constant or is lowered during the concentration step to retain selectivity at higher concentrations. Thus, “substantially constant TMP” refers to TMP versus membrane length, not versus filtration time.

Milk as a Feedstream

According to a preferred embodiment of the current invention, the TFF process employs three filtration unit operations that clarify, concentrate, and fractionate the product from a milk feedstream. This milk may be the product of a transgenic mammal containing a biopharmaceutical or other molecule of interest. In a preferred embodiment the system is designed such that it is highly selective for the molecule of interest. The clarification step removes larger particulate matter, such as fat globules and casein micelles from the milk feedstream. The concentration/fractionation steps remove most small molecules, including lactose, minerals and water, to increased purity and reduce volume of the product. The product of the TFF process is thereafter concentrated to a level suitable for optimal downstream purification and overall product stability. This concentrated product, containing the molecules of interest, is then aseptically filtered to assure minimal bioburden and enhance the stability of the molecules of interest for extended periods of time. According to a preferred embodiment of the current invention, the bulk product will realize a purity between 65% and 85% and may contain components such as goat antibodies, whey proteins (β Lactoglobulin, α Lactalbumin, and BSA), as well as low levels of residual fat and casein. This partially purified product is an ideal starting feed material for conventional downstream chromatographic techniques to further select and isolate the molecules of interest which could include, without limitation, a recombinant protein produced in the milk, an immunoglobulin produced in the milk, or a fusion protein.

Step # 1 (Clarification)

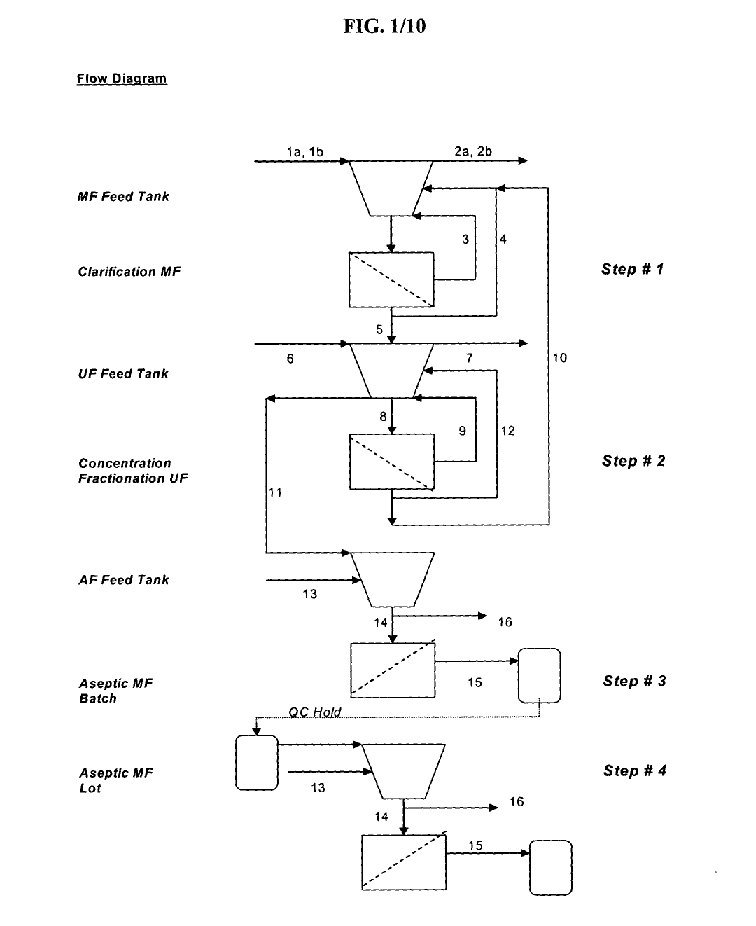

Turning to FIG. 1, transgenic mammal milk, preferably of caprine or bovine origin, is clarified utilizing batch-wise microfiltration. The milk is placed into a feed tank and pumped in a loop to concentrate the milk retentate two fold (see flow diagram in FIG. 1). Once concentrated the milk retentate is then diafiltered allowing the product and small molecular weight proteins, sugars, and minerals to pass through an appropriately sized membrane. According to the current invention, this operation is currently designed to take 2 to 3 hours and is will process 1000 liters of milk per day. The techniques and methods of the current invention can be scaled up and the overall volume of product that can be produced is dependent upon the commercial and/or therapeutic needs for a specific molecule of interest.

Step # 2 (Concentration/Fractionation)

Again referring to FIG. 1., the clarified permeate from the first step is concentrated and fractionated using ultrafiltration (“UF”). The clarified permeate flows into the UF feed tank and is pumped in a loop to concentrated the product two-fold. Once the concentration step is initiated the permeate from the UF is placed into the milk retentate in the clarification feed tank in the first step. The first and second step are sized and timed to be processed simultaneously. The permeate from the UF contains small molecular weight proteins, sugars, and minerals that pass through the membrane. Once 95% of the product is accumulated in the retentate of the UF, the clarification is stopped and a concentration/diafiltration of the UF material is begun. The product is concentrated 5 to 10 fold the initial milk volume and buffer is added to the UF feed tank. This washes away the majority of the small molecular weight proteins, sugars, and minerals. This operation is currently designed to take 2.5 to 3.5 hours and can process up to 500 liters of clarified permeate per day. As above, the techniques and methods of the current invention can be scaled up and the overall volume of product that can be produced is dependent via this concentration/fractionation process is dependent upon the commercial and/or therapeutic needs for a specific molecule of interest.

Step # 3 (Aseptic Filtration)

According to FIG. 1, and according to the current invention, the clarified bulk concentrate is then aseptically microfiltered. The resulting 50 to 100 liters of UF retentate is placed into a feed tank where it is pumped through a dead-end absolute 0.2 μm MF filtering system in order to remove the majority of the bioburden and enhance stability of the product for extended periods of time. The product is pumped through the filtering system of the invention and may then be directly filled into a final packaging configuration. Under conditions for processing a molecule of interest in a GMP facilities meeting clean room specifications (e.g., class 100 conditions) This operation is currently designed to take 0.5 to 1 hour and will process up to 100 liters of clarified bulk intermediate per day. As above, the techniques and methods of the current invention can be scaled up and the overall volume of product that can be produced is dependent via this concentration/fractionation process is dependent upon the commercial and/or therapeutic needs for a specific molecule of interest.

### EXAMPLE 1

### Milk as a Feedstream for the Production of a Molecule of Interest

The data below provides an application of the current invention that provides a membrane-based process to clarify, concentrate, and fractionate transgenically produced an IgG1 antibody from a raw milk feedstream. According to this example of the invention the transgenic mammal providing the milk for processing was a goat but other mammals may also be used including cattle, rabbits, mice as well sheep and pigs. Initial operational parameter ranges for processing were optimized utilizing CHO-cell produced IgG1 antibodies spiked into non-transgenic goat milk. When a transgenic goat capable of producing this molecule of interest came into lactation and began producing recombinant IgG1 antibodies in its milk, the several experiments were performed using CHO-cell produced recombinant IgG1 antibodies spiked into non-transgenic milk and were repeated with transgenic milk.

Pursuant to the current invention the experimental strategy was to determine the relationships between the filtration process variables that can be controlled on a large scale, (CM, V, TMP, T), where V is Flow Velocity, as can product passage, retention and quality. The relationships were established through a matrix of individual bench scale experiments, and optimal windows of operation were identified. These optimal parameters are combined into a “Dual TFF” experimental series where overall yield and mass balance are investigated. Performance was determined by product yield, clarity, and flux efficiency. The following process variables are investigated in the individual bench scale experimental matrix.

Concentration (Cm)

Optimal milk concentration factors were be determined with empirical product passage data. The rate of product passage per meter squared in a fixed time is referred to as the product flux (Jp). Product flux will be measured in relationship to concentration factor during the Clarification step (Unit Operation # 1).

Again referring to FIG. 1, below is provided an explanation of the elements of the invention.

FIG. 1 Elements

Process Stream Description

| Stream | |
|---|---|
| Number | Description |
| 1a | Raw tg Milk |
| 1b | Microfiltration CIP Solutions |
| 2a | Microfiltration Retentate to drain after Diafiltration |
| 2b | Used CIP Solutions to drain |
| 3 | In process MF Retentate (loop) |
| 4 | MF CIP Recirculation (loop) |
| 5 | Microfiltration Filtrate |
| 6 | Ultrafiltration CIP Solutions |
| 7 | Used CIP Solutions to drain |
| 8 | Ultrafiltration Feed (Microfiltration Filtrate) |
| 9 | In process UF Retentate (loop) |
| 10 | Ultrafiltration Permeate (To Diafilter MF Retentate) |
| 11 | Concentrated Clarified Bulk |
| 12 | UF CIP Recirculation (loop) |
| 13 | AF CIP Solutions |
| 14 | Aseptic Filter Feed |
| 15 | Bioburden Reduced Concentrated Clarified Bulk |
| 16 | Used CIP Solutions to drain |

In its broadest aspect, the high-performance tangential-flow filtration process contemplated by the invention provided herein involves passing the mixture of the species to be separated through one or more filtration membranes in an apparatus or module designed for a type of tangential-flow filtration under certain conditions of TMP and flux. The TMP is held at a range in the pressure-dependent region of the flux v. TMP curve, namely, at a range that is no greater than the TMP value at the transition point. Thus, the filtration is operated at a flux ranging from about 5% up to 100% of transition point flux. See Graphs. A and B below, wherein the flux v. TMP curve is depicted along with the transition point. As a result, the species of interest are selectively retained by the membrane as the retentate while the smaller species pass through the membrane as the filtrate, or the species of interest pass through the membrane as the filtrate and the contaminants in the mixture are retained by the membrane. It should be noted that the species of interest for ultrafiltration preferably are biological macromolecules having a molecular weight of at least about 1000 daltons, and most preferably polypeptides and proteins. Also preferred is that the species of interest be less than ten-fold larger than the species from which it is to be separated, i.e., contaminant, or be less than ten-fold smaller than the species from which it is to be separated.

As used herein, the expression “means for re-circulating filtrate through the filtrate chamber parallel to the direction of the fluid in the filtering chamber” refers to a mechanism or apparatus that directs a portion of the fluid from the filtrate chambers to flow parallel to and in substantially the same direction (allowing for some eddies in flow to occur) as the flow of fluid passing through the adjacent filtering chamber from the inlet to the outlet of the filtering chamber. Preferably, this means is a pumping means.

It is noted that the TMP does not increase with filtration time and is not necessarily held constant throughout the filtration. The TMP may be held approximately constant with time or may decrease as the filtration progresses. If the retained species are being concentrated, then it is preferred to decrease the TMP over the course of the concentration step.

Each membrane preferably has a pore size that retains species with a size of up to about 10 microns, more preferably 1 kDa to 10 microns. Examples of species that can be separated by ultrafiltration include proteins, polypeptides, colloids, immunoglobulins, fusion proteins, immunoglobulin fragments, mycoplasm, endotoxins, viruses, amino acids, DNA, RNA, and carbohydrates. Examples of species that can be separated by microfiltration include mammalian cells and microorganisms such as bacteria.

Because membrane filters are not perfect and may have holes or irregularities that may be large enough to allow some intended retentate molecules to slip through, a preferred aspect herein is to utilize more than one membrane having the same pore size, where the membranes are placed so as to be layered parallel to each other, preferably one on top of the other. Preferably the number of membranes for this purpose is two.

While the flux at which the pressure is maintained in the above process suitably ranges from about 5 to 100%, the lower the flux, the larger the surface area of the membrane required. Thus, to minimize membrane cost, it is preferred to operate at a pressure so that the flux is at the higher end of the spectrum. The preferred range is from about 50 to 100%, and the more preferred range is about 75 to 100%, of the transition point flux.

While the TMP need not be maintained substantially constant along the membrane surface, it is preferred to maintain the TMP substantially constant. Such a condition is generally achieved by creating a pressure gradient on the filtrate side of the membrane. Thus, the filtrate is recycled through the filtrate compartment of the filtration device in the same direction and parallel to the flow of the mixture in the retentate compartment of the device. The inlet and outlet pressures of the recycled material are regulated such that the pressure drop across the filtrate compartment equals the pressure drop across the retentate compartment.

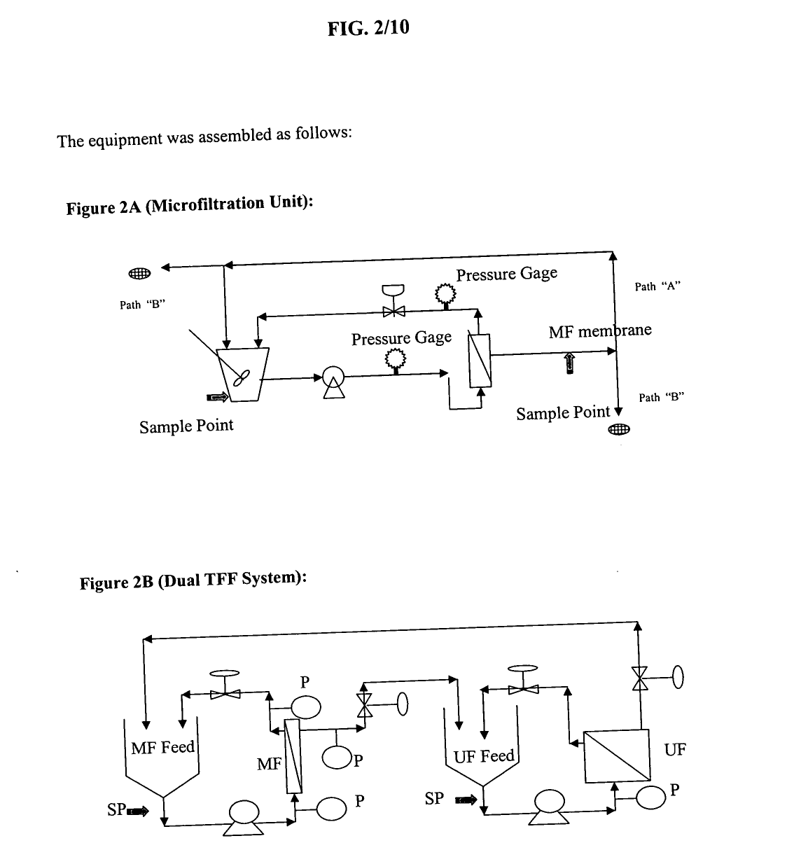

Several practical means can be used to achieve this filtrate pressure gradient. Some examples of preferred embodiments are the configurations shown in FIGS. 2A and 2B. According to these configurations the solutes to be separated enter the device through an inlet conduit 36, which communicates with a fermenter tank (not shown) if the products to be separated are in a fermentation broth. It may also communicate with a vessel (not shown) that holds a source of transgenic (Tg) milk or cell lysate or a supernatant after cell harvest in cell culture systems. The flow rate in conduit 36 is regulated via a pumping means 40. The pump is any suitable pump known to those skilled in the art, and the flow rate can be adjusted in accordance with the nature of the filtration as is known to those skilled in the art.

In a Microfiltration Unit 30 of the current invention, a pressure gauge 45 is optionally employed to measure the inlet pressure of the flow from the pumping means 40. The fluid in inlet conduit 36 enters filtration unit 50. This filtration unit 50 contains a filtering chamber 51 in an entrance top portion thereof and a filtrate chamber 52 in the exit portion. These two compartments are divided by a filtration membrane 55. The inlet fluid flows in a direction parallel to filtration membrane 55 within filtering chamber 51. The upper, filtering chamber 51 receives the mixture containing the solute containing a molecule of interest of interest. Molecules small that the target molecule are able to pass through the membrane 55 into the filtrate or exit chamber 52. The concentrated retentate passes from the filtration unit 50 via outlet conduit 60, where it may be collected and processed further by a microfiltration (MF) membrane 65, if necessary, to obtain the desired species of interest including moving through an additional membrane. During this entire process, and for quality control purposes, a series of sample points 99 are contemplated by the current invention to allow monitoring of molecule concentration, pH and contamination—“path B”. Alternatively, a retentate stream is circulated back to a tank or fermenter 35 “path A” from whence the mixture originated, to be recycled through inlet conduit 36 for further purification.

A solution containing molecules of interest that pass through the membrane 55 into the filtrate chamber 52 can also leave filtration unit 50 via outlet conduit 70 at the same end of the filtration unit 50 as the retentate fluid exits via outlet conduit 60. However, the solution and molecules of interest flowing through outlet conduit 70 are sent back to tank 35, and are measured by pressure gage 72 for further processing.

Similarly, and as depicted in FIG. 2B a Dual TFF system 80 according to the current invention is contemplated.

In the configuration shown in FIG. 2A, the membranes will need to be placed with respect to chambers 51 and 52 to provide the indicated flow rates and pressure differences across the membrane. The membranes useful in the process of this invention are generally in the form of flat sheets, rolled-up sheets, cylinders, concentric cylinders, ducts of various cross-section and other configurations, assembled singly or in groups, and connected in series or in parallel within the filtration unit. The apparatus generally is constructed so that the filtering and filtrate chambers run the length of the membrane.

Suitable membranes are those that separate the desired species from undesirable species in the mixture without substantial clogging problems and at a rate sufficient for continuous operation of the system. Examples include microporous membranes with pore sizes typically from 0.1 to 10 micrometers, and can be made so that it retains all particles larger than the rated size. Preferably they are ceramic for both microfiltration uses and TFF uses according to the current invention. Ultrafiltration membranes have smaller pores and are characterized by the size of the protein that will be retained. They are available in increments from 1000 to 1,000,000 Dalton nominal molecular weight limits.

Ultrafiltration membranes are most commonly suitable for use in the process of this invention. Ultrafiltration membranes are normally asymmetrical with a thin film or skin on the upstream surface that is responsible for their separating power. They are commonly made of regenerated cellulose or polysulfone.

Membrane filters for tangential-flow filtration system 80 are available as units of different configurations depending on the volumes of liquid to be handled, and in a variety of pore sizes. Particularly suitable for use in the present invention, on a relatively large scale, are those known, commercially available tangential-flow filtration units.

In an alternative and preferred apparatus, and for the reasons presented above, the microfiltration unit 30 of FIG. 2A comprises multiple, preferably two, filtration membranes, as membranes 56 and 57, respectively. These membranes are layered in a parallel configuration.

The invention also contemplates a multi-stage cascade process wherein the filtrate from the above process is passed through a filtration membrane having a smaller pore size than the membrane of the first apparatus in a second tangential-flow filtration apparatus, the filtrate from this second filtration is recycled back to the first apparatus, and the process is repeated.

One tangential-flow system 80 suitable for process according to the invention or use in conjunction with a microfiltration unit 30 is shown in FIG. 2B. Here, a first vessel 85 is connected via inlet conduit 90 to a filtering chamber 96 disposed within a filtration unit 95. A first input pumping means 100 is disposed between the first vessel 85 and filtering chamber 96. The filtering chamber 96 is connected via an outlet conduit 110 to the first vessel 85. The filtering chamber 96 is separated from a first filtrate chamber 97 situated directly below it within filtration unit 95 by a first filtration membrane 115. The first filtrate chamber 97 has an outlet conduit 98 connected to the inlet of chamber 97 with a filtrate pumping means 120 disposed in the conduit 98. Conduit 45, which is connected to outlet conduit 98, is connected also to a second vessel 120.

This vessel 120 is connected via inlet conduit 125 to a second filtering chamber 127 disposed within a second filtration unit 130. A second input pumping means 133 is disposed between the second vessel 120 and filtering chamber 127. The filtering chamber 127 is separated from the second filtrate chamber 129 situated directly below it within filtration unit 130 by a second filtration membrane 128. The second filtrate chamber 129 has an outlet conduit 135 connected to the inlet of chamber 129 with a filtrate pumping means 140 disposed in the conduit 135. Conduit 125, which is connected to outlet conduit 135, is connected also to a third vessel 150.

This vessel 150 is connected via inlet conduit 155 to a third filtering chamber 157 disposed within a third filtration unit 160. A third input pumping means 165 is disposed between the third vessel 150 and filtering chamber 157. The filtering chamber 157 is separated from the third filtrate chamber 159 situated directly below it within filtration unit 160 by a third filtration membrane 165. The third filtrate chamber 159 has an outlet conduit 170 connected to conduit 155, which is connected to first vessel 150, to allow the filtrate to re-circulate to the original tank. Sample points 99 were also provided for monitoring the process, as well as pressure gages 175.

The process of the present invention is well adapted for use on a commercial scale. It can be run in batch or continuous operations, or in a semi-continuous manner, e.g., on a continuous-flow basis of solution containing the desired species, past a tangential-flow filter, until an entire large batch has thus been filtered, with washing steps interposed between the filtration stages. Then fresh batches of solution can be treated. In this way, a continuous cycle process can be conducted to give large yields of desired product, in acceptably pure form, over relatively short periods of time.

The unique feature of tangential-flow filtration as described herein with its ability to provide continuous filtration of solids-containing solutions without filter clogging results in a highly advantageous process for separating and purifying biological reaction products for use on a continuous basis and a commercial scale. Moreover, the process is applicable to a wide range of biological molecules, e.g., protein products of transgenic origin, antibodies, cell fragments and cell culture lysates.

The following examples illustrate the invention in further detail, but are not intended to be limiting. In these examples, the disclosures of all references cited are expressly incorporated by reference.

Materials and Methods

For all experiments conducted with the microfiltration system except a feed-and-bleed experiment, the equipment used was the following:

60 lpm pump calibrated to correlate pump (Pump Curve)

1″ OD stainless steel sanitary piping

0.2 um pore size ceramic membrane of either 0.2 sqft or 1.5 sqft

Stainless steel sanitary membrane holder with one ½″ outlet port

¼″ ID flexible permeate tubing

Diaphragm valve on the retentate line

2 pressure gauges

Steel 1.2 L feed reservoir

¾″ ID flexible retentate tubing.

For all dual TFF experiments, the preceding equipment was coupled with the following equipment:

Diaphragm pump with maximum output of 800 mLPM

¼″ ID flexible pressure resistant tubing on all lines

1 pressure gauge for feed pressure measurements

2 diaphragm valves on the retentate and permeate lines

30 kDa NMWCO PES Pall Filtron Centramate membrane of either 0.2 sqft or 1 sqft

Stainless steel Pall Filtron Centramate membrane holder

1 stainless steel u-bend pipe to connect permeate ports.

Membrane Selection

The membranes selected for the dual TFF system were selected from a group of membranes of varying geometries and nominal molecular weight cut-offs. Previous studies explored the use of polymeric based high MWCO UF membranes, as well as ceramics, for the clarification step. Concentrating the milk down 2× and then doing dual TFF challenged all membranes. The membranes were then analyzed for reusability by challenging them with multiple runs and cleanings. A membrane was considered recovered for the next process when the normalized water flux was maintained above 80% of the virgin membrane. None of the flat sheet polymeric membrane cassettes maintained the target water flux recovery after 3 uses, while the ceramic membrane was recovered more than 60 times. This was due to the ability to clean the ceramic using harsher conditions of higher chemical concentration and higher temperatures. The 30 kDa ultrafiltration membrane maintained high water flux recoveries beyond 20 cycles.

The first unit, used to clarify the milk and pass the IgG1 antibody, was tested using 0.2 um nominal ceramic tubular membranes. The second system used to capture the IgG1 antibody was tested with flat sheet ultrafiltration membranes of 30 kDa molecular weight cut-offs.

Analytical Methods

Samples from each experiment samples were analyzed for IgG content by protein A HPLC, for degradation by SDS-PAGE, for modification by isoelectric focusing (IEF), and for aggregation by size exclusion chromatography (SEC).

Procedure

A series of controlled experiments were conducted employing 0.2 μm molecular weight cut-off ceramic microfiltration membranes in the hopes of understanding process operational relationships. Product Flux (Jp) was measured as it related to flow velocity (u), trans-membrane pressure (TMP), temperature (t), and milk concentration (c). Once relationships were established, optimal windows of operation were determined and a compiled process was tested. Samples were taken and mass balance data was gathered and analyzed for initial product yield and throughput. (Please see, FIGS. 2A and 2B).

Temperature Experiment

The objective was to determine the range of operating temperatures which give optimum IgG1 antibody flux at lowest volume through a 0.2 um, 3 mm channel ceramic MF membrane. To analyze IgG1 antibody degradation by SDS-PAGE and Western blot during processing the pH of each milk segment was taken prior to milk pooling. The milk is pooled into the MF feed tank and total volume is recorded. The MF pump controller is ramped up from 20 Hz to 45 Hz (approximately 5 L/min to approximately 20 L) at this time. All parameters at every successive time point are recorded such as temperature, pressures, cross-flow rate, permeate flow rate, and volume. This MF loop is run in recirculation (path A) for 5 minutes. The transmembrane pressure is adjusted to 12 psig and re-circulated (path A) for 5 minutes (Maintained a temperature of 20° C.). The permeate line is directed to drain until milk was concentrated 2× the original milk volume (permeate was collected). Temperature was maintained at 20° C. Samples 2 and 3 were taken from the feed reservoir and from the permeate line. The permeate line was then returned to path A and re-circulated for 10 minutes. Samples 4 and 5 were taken. Temperature was allowed to increased to 25° C. The system then re-circulated for 10 minutes and samples 6 and 7 were taken. Temperature was allowed to increased to 30° C. The system then re-circulated for 10 minutes and samples 8 and 9 were taken. Temperature was allowed to increased to 35° C. The system then re-circulated for 10 minutes and samples 10 and 11 were taken. Temperature was allowed to increased to 40° C. The system then re-circulated for 10 minutes and samples 12 and 13 were taken. The pump was then turned off and samples were stored at 2-8° C. and sent for IgG quantitation (SDS-PAGE of samples 1,3,5,7,9,11, 13, 15, 17 for degradation and aggregation. Samples 1 and 16 were analyzed by IEF. Samples 1, 3, 16, 17 were analyzed by SEC).

MF Milk Concentration Experiment

The objective of this experiment according to a preferred embodiment of the invention was to determine the range of initial milk concentration which gives optimum IgG1 antibody flux at lowest volume through a 0.2 um, 3 mm channel ceramic MF membrane.

In terms of procedure the pH of each milk segment was taken prior to milk pooling. The milk is pooled into the MF feed tank and total volume is recorded. The MF pump controller is ramped up from 20 Hz to 45 Hz (approximately 5 L/min to approximately 20 L) at this time. All parameters at every successive time point are recorded such as temperature, pressures, cross-flow rate, permeate flow rate, and volume. This MF loop is run in recirculation (path A) for 5 minutes. The transmembrane pressure is adjusted to 12 psig and re-circulated (path A) for 5 minutes (Maintained a temperature of 20° C). Adjusted transmembrane pressure to 15 psig and re-circulated (path A) for 5 minutes. The permeate line was directed to drain until milk was concentrated, and 550 ml of permeate was collected, then returned the permeate line to path A.(Re-circulated for 10 minutes) Samples 2 and 3 were taken from the feed reservoir and the permeate line respectively.

The permeate line was directed to path B and 600 ml of milk was added to the feed reservoir. The permeate line was directed to drain until milk was concentrated, and 500 ml of permeate was collected, then returned the permeate line to path A. (Re-circulated for 10 minutes) Samples 4 and 5 were taken from the feed reservoir and the permeate line respectively. The permeate line was then directed to path B and 500 ml of milk was added to the feed reservoir. The permeate line was directed to drain until milk was concentrated, and 500 ml of permeate was collected, then returned the permeate line to path A.(Re-circulated for 10 minutes) Samples 6 and 7 were taken from the feed reservoir and the permeate line respectively. The permeate line was then directed to path B and 380 ml of milk was added to the feed reservoir. The permeate line was directed to drain until milk was concentrated, and 400 ml of permeate was collected, then returned the permeate line to path A.(Re-circulated for 10 minutes) Samples 8 and 9 were taken from the feed reservoir and the permeate line respectively. The pump was then turned off. Samples were stored at 2-8° C. and sent for IgG quantitation by protein A analysis, SDS-PAGE and Western for degradation and aggregation, SEC for aggregation, and IEF for isoelectric point shifts.

Flow Velocity and TMP Experiment

To determine the relationship between trans-membrane pressure (TMP), cross-flow velocity, filtrate clarity, membrane liquid flux, and passage of IgG1 antibody through a 0.2 um, 3 mm channel ceramic MF membrane. One liter of non-transgenic milk spiked with 3.7 g of IgG1 antibody (2.5 g/L) is placed into a 1.5 liter feed tank. The spiked milk is continuously agitated at room temperature as it is pumped through the MF loop at 30 L/min with the following initial parameters:

| | |
|---|---|
| Membrane Area | 0.164 sqft |
| Membrane Pore Size | 0.20 um |
| Initial Milk Vol. | 1.0 L |
| Feed Pressure | 10 psig |
| Permeate Pressure | 0 psig |
| Feed Flow Rate | 20 L/min |
| Milk Temp. | 30° C. |

Sample #1

This sample was taken from milk spiked milk. The permeate line of the MF is fed to the feed reservoir. At time equals 10 min the permeate is directed through path “B” (permeate to drain). This will concentrate the milk to 500 ml. Once the milk is 2× the original concentration, the permeate is switched back to path “A” (re-circulation back to feed reservoir). After 10 min in re-circulation, sample numbers 2 and 3 are taken then the back pressure valve is adjusted to cause the feed pressure near the pump to read 10 psi. Feed flow rate is maintained at 13.35 l/min by adjusting the pump speed to 55 Hz. After 10 min in re-circulation, sample numbers 4 and 5 are taken and the back pressure valve is adjusted to cause the feed pressure near the pump to read 14 psi. Feed flow rate is maintained at 13.35 L/min by adjusting the pump speed to 60.66 Hz. After 10 min in re-circulation sample numbers 6 and 7 are taken and the back pressure valve is adjusted to cause the feed pressure near the pump to read 12 psi. Feed flow rate is adjusted to 7.75 L/min by adjusting the pump speed to 40 Hz. After 10 min in re-circulation sample numbers 8 and 9 are taken then the back pressure valve is adjusted to cause the feed pressure near the pump to read 14 psi.

Feed flow rate is maintained at 7.75 l/min by adjusting the pump speed to 43.45 Hz. After 10 min in re-circulation, sample numbers 10 and 11 are taken and the back pressure valve is adjusted to cause the feed pressure near the pump to read 10 psi. Feed flow rate is adjusted to 12.36 L/min by adjusting the pump speed to 48 Hz. After 10 min in re-circulation sample numbers 12 and 13 are taken and the back pressure valve is adjusted to cause the feed pressure near the pump to read 14 psi. Feed flow rate is maintained at 12.36 L/min by adjusting the pump speed to 55.44 Hz. After 10 min in re-circulation sample numbers 14 and 15 are taken then the back pressure valve is adjusted to cause the feed pressure near the pump to read 20 psi. Feed flow rate is adjusted to 12.36 l/min by adjusting the pump speed to 61.69 Hz. After 10 min in re-circulation, sample numbers 16 and 17 are taken and the feed flow rate is adjusted to read 13.35 L/min, 64.64 Hz, and the back pressure valve is adjusted to cause the feed pressure near the pump to read 20 psi. After 10 min in recirculation, sample numbers 18 and 19 are taken and the feed flow rate is adjusted to 7.75 L/min by adjusting the pump speed to 52.65 Hz and the back pressure valve is adjusted to cause the feed pressure near the pump to read 20 psi. After 10 min in re-circulation sample numbers 20 and 21 are taken and the pump is turned off, and the pump is turned off. All samples are refrigerated and analyzed by protein A assay for total IgG content. The permeate samples (3, 5, 7, 9, 11, 13, 15, 17, 19, 21) will be visually inspected for clarity.

Dual Process Experiment

To test the process parameters determined in previous experiments on dual TFF system to recover cell culture IgG1 antibody from non-transgenic milk. Non-transgenic milk was spiked with 2.4 g of cell culture IgG1 antibody for a total volume of 1000 ml and sample number 1 was taken. The spiked milk was placed in the feed reservoir of the microfiltation system and pumped across the membrane at 13.4 l/min. The temperature, pressures, permeate flow rates and volume were recorded at each subsequent time point. The system was adjusted to the following initial parameters:

| | |
|---|---|
| Membrane Area: | 0.2 sqft |
| Membrane Pore Size: | 0.20 um |
| Initial Milk Vol.: | 1000 mL |
| Transmembrane Pressure | 14 psig |
| Permeate Pressure | 0 psig |
| Concentration | 1 x |

The permeate line of the MF was fed to the feed reservoir. At time equals 10 min the permeate was directed through path “B” (permeate to drain). Once the milk was 2× the original concentration or 500 ml, the permeate was switched back to path “A” (re-circulation back to feed reservoir). The UF pump was started up at the following initial conditions:

| | |
|---|---|
| Membrane Area: | 0.2 sqft |
| Membrane Pore Size: | 30 kDa |
| Initial Volume: | 500 mL |
| Transmembrane Pressure | 7.3 psig |
| Permeate Pressure | 1.4 psig |
| Concentration | 1 x |

After 10 minutes in recirculation, the retentate and permeate pressures of the UF were adjusted such that the permeate flow rate of the UF equaled the permeate flow rate of the MF. The permeate line of the UF was then directed to the feed reservoir of the MF and the permeate line of the MF was directed to the feed reservoir of the UF, beginning diafiltration. The system was run for a total of 326 minutes and samples were taken of each diavolume. All samples were refrigerated and analyzed by protein A assay for total IgG content and SEC for aggregation.

Experiments using CHO-cell produced IgG1 antibody showed the optimum flow velocity to be approximately 23 cm/s at a trans-membrane pressure of 14 psig (Graph #C & D). However, the feedstream containing the protein or immunoglobulin of interest could be from any source capable of producing such a molecule, including without limitation, transgenic animals producing exogenously derived recombinant proteins. Optimal temperature, according to the current invention, was between 30-35° C. (Graph # A). Non-transgenic milk showed liquid flux to be highest at 1.5-2× (Graph # B). When these parameters were tested in a dual TFF system, 82.3% yield was obtained (Graph # H). The flow velocity and trans-membrane pressure experiment was repeated using natural transgenic milk from goat C1017 and showed the optimal flow velocity to be between 40-45 cm/s at a trans-membrane pressure of 16 psig (Graph #E & F). The dual TFF process test conducted on natural transgenic milk at the parameters discovered using CHO-cell IgG1 antibody gave a yield of 64% (Graph #G). The source of transgenic goat could be from any mammal, preferably from an ungulate, and most preferably caprine or bovine in origin.

[image]

[image]

Though spiking CHO-cell IgG1 antibody into non-transgenic milk gave an initial look at the behavior of IgG1 antibody in milk, naturally lactated milk containing the IgG1 antibody required different optimization parameters for the use of a 0.2 mm ceramic membranes preferably used according to the current invention. The spiking study showed a lower optimum flow velocity and very high product fluxes than the transgenic milk study. Moreover, running the dual TFF system using the parameters optimized for transgenic milk provided lower product recovery for natural non-transgenic milk gave spiked with the IgG1 antibody.

Tangential flow filtration (TFF) was implemented as a process to clarify and stabilize IgG1 antibody in a milk matrix by removing particulate matter such as fat, casein micelles, and bacteria from raw milk. TFF is widely used in both the biotechnology and dairy industries to remove impurities and concentrate product. In order to use TFF effectively according to the current invention it is important that the proper membranes are chosen, the process parameters (temperature, trans-membrane pressure, cross-flow velocity, and milk concentration) are optimized for high product flux, and the cleaning and storage procedures were developed to ensure long membrane life. Experimental matrix parameters are described herein, according to the current invention and applied to transgenic goat milk to confirm previous operational parameters. Membrane cleaning and storage conditions were also investigated. An aseptic filtration step was developed to remove any bacteria remaining from the clarified milk product containing a protein of interest after the TFF process is complete. Process information was then transferred to pilot scale equipment were initial engineering runs were conducted. Some process design criteria included, using no additives to prevent the need for water for injection, long membrane life, high yield, and short processing time. The process of the current invention was preferably designed to be scalable for pilot and manufacturing operations.

Process Description

To perform dual TFF using a ceramic 0.2 μm microfiltration membrane and a 30 kDa ultrafiltration membrane to clarify and concentrate transgenic goat milk from goat D035, the system was sanitized with 0.1M sodium hydroxide. Then the milk must be pooled and raised to 15-20° C. The milk must be concentrated to half of its' original volume on the microfiltration system by collecting the permeate of the ceramic membrane. The MF must be run at 14 lpm cross flow rate with 15 psi of transmembrane pressure. The temperature must be held near or at 27C. The ultrafiltration system must then be started up at 1.6-2 LPM cross flow rate. The retentate and permeate pressures of the UF must be adjusted to cause the permeate flow rate to match the permeate flow rate of the MF. Once the UF permeate flow rate matches that of the MF, the two systems must be coupled such that the permeate line of the MF is directed to the feed reservoir of the UF, and the permeate line of the UF is directed to the feed reservoir of the MF. The systems should be run coupled for 5-6 diafiltration volumes. Once diafiltration is complete, the systems are disconnected, the MF is shut of, drained and cleaned, and the UF permeate is directed to drain until the volume of bulk clarified concentrate in the feed reservoir of the UF is concentrated to half it's volume for a total concentration of 4×. The UF is then drained, the bulk clarified concentrate is aseptically filtered, and the UF is cleaned. Both systems are stored in 0.1M sodium hydroxide. A process diagram is provided in FIG. 1.

Membrane Selection

Based on previous studies, the first unit, used to clarify the milk and pass the IgG1 antibody, was tested using 0.2 um nominal ceramic tubular membranes. The second system used to capture the IgG1 antibody was tested with flat sheet ultrafiltration membranes of 30 kDa molecular weight cut-offs. Samples from each experiment using D035 milk were analyzed for IgG content by protein A HPLC, for degradation by SDS-PAGE, for modification by isoelectric focusing (IEF), and for aggregation by size exclusion chromatography (SEC). The range of initial milk concentration that gave optimum IgG1 antibody flux at lowest volume through a 0.2 um, 3 mm channel ceramic MF membrane was determined. IgG1 antibody degradation was analyzed by SDS-PAGE and Western blot during processing to determine the effects (if any) of the concentration step on the IgG1 antibody. Two experiments were completed to investigate milk concentration, which included one non-transgenic milk run and one transgenic milk run.

Non-Transgenic Feed-And-Bleed Experiment

Non-transgenic milk was used to analyze liquid flux decay during concentration using the 0.2 um ceramic microfiltration membrane since an abundant supply of non-transgenic milk is available. The equipment used for this experiment included the same equipment described for microfiltration experiments, but it was supplemented by a second feed reservoir and a feed pump to flow milk into the feed reservoir of the microfiltration system at the same rate that permeate was flowing out of the membrane. The equipment schematic is:

[image]

As seen in Graph I, the feed reservoir was filled with 1500 ml of milk and the pump was started at 45 Hz. The system was run in re-circulation for 10 minutes with no retentate pressure. All parameters were recorded. The retentate pressure was then increased to 10 psig for a transmembrane pressure of 11 psig. This transmembrane pressure was held constant throughout the experiment by adjusting the retentate valve. The permeate was sent to drain, and a second pump was started up to pump fresh milk into the feed reservoir at the same rate as permeate was removed, keeping the volume in the feed reservoir constant. All parameters were recorded at 5-10 minute intervals, and the second pump speed was adjusted to keep the level of milk in the feed reservoir constant. The experiment was run until the milk was concentrated 5.37× or 82%.

Transgenic Milk Concentration vs. Product Flux

The pH and volume of each milking of D035 were measured and recorded in the pH chart. Segments D035 RM01-001PD through RM-004PD were pooled for a total volume of 3 L. Sample F1 was taken of the pool, and 1500 L were poured into the feed reservoir. The pump was started up and the speed was increased from 20 Hz to 45 Hz (approximately 5 LPM to approximately 20 L). Temperature, pressures, cross-flow rate, permeate flow rate, and volume were recorded. All parameters were recorded at every successive time point. The system was run in recirculation (path A) for 5 minutes. The transmembrane pressure was adjusted to 12 psig and re-circulated (path A) for 5 minutes. The permeate line was directed to drain until the milk was concentrated 1.5×, 550 ml of permeate was collected. Samples F2 and P1 were taken of the feed reservoir and of the collected permeate. The permeate line was returned to path A and re-circulated for 10 minutes. Samples F3 and P2 were taken of the feed reservoir and permeate line respectively. Thereafter 500 ml of fresh milk was added to the feed reservoir, and the permeate was then directed to path B to concentrate the milk down to 2× by collecting 500 ml more. The permeate line was returned to path A and re-circulated for 10 minutes. Samples F4 and P3 were taken. This was repeated to concentrate the milk down to 2.5× and 3× and the sampling continued. The pump was turned off. Samples were stored at 2-8° C. and sent for IgG quantitation and SDS-PAGE analysis.

The range of operating temperatures that gave optimum IgG1 antibody flux at lowest volume through a 0.2 um, 3 mm channel ceramic MF membrane were determined. IgG1 antibody degradation due to processing was analyzed by SDS-PAGE. Isoform modification was tracked by IEF. The pH and volume of each milking of goat D035 were measured and recorded in the pH chart. Segments D035 RM01-005PD-D035 RM01-008PD were pooled for a total volume of 3000 ml. Sample number F1 was taken of the pool. 2L were poured into the feed reservoir. The pump was started up and the speed was increased from 20 Hz to 45 Hz (approximately 5 LPM to approximately 20 L). Temperature, pressures, cross-flow rate, permeate flow rate, and volume were recorded. All parameters were recorded at every successive time point. The system was run in recirculation (path A) for 5 minutes. The transmembrane pressure was adjusted to 12 psig and re-circulated (path A) for 5 minutes. The temperature was maintained at 20 C. The permeate line was directed to drain until the milk was concentrated 2.5×, 800 ml of permeate was collected. Samples F2 and P1 were taken of the feed reservoir and of the collected permeate. The permeate line was returned to path A and allowed to re-circulate, the TMP was reduced to 2 psi, and the pump speed was decreased to 28 Hz (17 LPM) for 17 minutes to allow the temperature to drop to 23° C. The pump speed and TMP were increased to 45 Hz and 15 psi respectively, and allowed to recirculate for 5 mm to 24° C. Samples F3 and P2 were taken. The temperature was allowed to increase to 27 C and the milk was re-circulated for 5 minutes. Samples F4 and P3 were taken. This was repeated for 29° C. and 36° C. The remainder of the fresh milk was clarified through the MF membrane. The pump was turned off. Samples were stored at 2-8 C and sent for IgG quantitation, IEF, and SDS-PAGE analysis.

Flow Velocity and TMP vs. Product Flux

The range of transmembrane pressures (TMP) and cross-flow velocities which gave optimum IgG1 antibody flux through a 0.2 um, 3 mm channel ceramic MF membrane were determined. The pH and volume of each segment of D035 were measured and recorded in the pH chart. Segments D035 RM01-009PD-D035 RM01-0012PD were pooled for a total volume of 3700 ml. Sample F1 was taken of the pool. 1 L was poured into the feed reservoir. The pump was started up and the speed was increased from 20 Hz to 45 Hz (approximately 5 LPM to approximately 20 L). Temperature, pressures, cross-flow rate, permeate flow rate, and volume were recorded. All parameters were recorded at every successive time point. The system was run in recirculation (path A) for 5 minutes. The transmembrane pressure was adjusted to 12 psig and re-circulated (path A) for 5 minutes. The permeate line was directed to drain until the milk was concentrated 2×, 500 ml of permeate was collected. Samples F2 and P1 were taken of the feed reservoir and of the collected permeate. The permeate line was returned to path A and allowed to recirculate for 10 minutes. Samples 4 and 5 were taken. The transmembrane pressure was adjusted to 10 psig and maintained the feed flow rate by increasing the pump speed to 38.81 Hz (˜14 LPM). The milk was re-circulated through the MF system for 10 minutes and then sample P3 was taken. This procedure was repeated according to the chart below:

| Retentate | | | |
|---|---|---|---|
| Flow Rate | Pump | Transmembrane | |
| (LPM) | Speed (Hz) | Pressure (psig) | Sample (ID) |
| 14 | 38.81 | 10 | P2 |
| 14 | 45.86 | 15 | P3 |
| 12 | 35.96 | 10 | P4 |
| 12 | 47.03 | 15 | P5 |
| 8 | 30.26 | 10 | P6 |
| 8 | 37.63 | 15 | P7 |
| 16 | 41.66 | 10 | P8 |
| 16 | 48.37 | 15 | P9 |
| 16 | 54.19 | 20 | P10 |
| 14 | 51.44 | 20 | P11 |
| 12 | 48.68 | 20 | P12 |
| 8 | 43.17 | 20 | P13 |

The pump was turned off. Samples were stored at 2-8° C. and sent for IgG quantitation by protein A quantitation, IEF, and SDS-PAGE analyses.

Process Test

To clarify D035 milk using dual TFF with a 0.2 um ceramic MF of 1.5 sqft feeding a 30 kDa UF of 1.0 sqft, and analyze the recovery of IgG1 antibody at each diafiltration volume. The pH and volume of each milk segment from goat D035 was recorded in pH chart. The segments D035 RM01-029PD-RM01-032PD for 1776-032601-01-B and RM01-033PD-RM01-036PD were pooled for a total volume of about 4 L for each experiment. Sample number F1 was taken of the pool. 1500 mL was poured into the feed reservoir. The pump was started, and the speed was ramped up from 20 Hz to 45 Hz (approximately 5 LPM to approximately 20 L). Recorded temperatures, pressures, MF cross-flow rate, permeate flow rates, and volume. Recorded all parameters at every successive time point. Ran in recirculation (path A) for 5 minutes. Adjusted transmembrane pressure to 15 psig and re-circulated (path A) for 5 minutes. The permeate line was directed to a graduated cylinder. Added fresh milk to feed reservoir as the volume declined. The permeate was collected until the milk was concentrated to 3×, and 2770 ml was collected. Samples F2 and P1 were taken from the MF feed reservoir and of the collected permeate. The permeate was again directed to path A. The cross flow rate was increased to 14 LPM with the transmembrane pressure at 15 psig. The UF was started with a cross flow rate of 0.8 LPM and 11 psi feed pressure. Each system was simultaneously run in recirculation for 10 min. The permeate of the UF was directed to drain, and 800 ml of permeate was collected. The permeate flow rate of the MF was measured. The retentate and permeate pressures on the UF were adjusted to produce a permeate flow rate equal to that of the MF. The permeate of the MF was directed to the feed reservoir of the UF, and the permeate of the UF was directed to the feed reservoir of the MF. The diafiltration time was calculated (refer to the calculations section). Took samples at the conclusion of each diafiltration. Measured permeate flow rates and recalculated the diafiltration time. Performed 7 diafiltration volumes. Disconnected the two systems and turned off the MF. Directed the permeate of the UF to drain and concentrated the clarified milk down to a total concentration of 4×. The UF was then turned off. Samples were stored at 2-8° C. and sent for IgG quantitation, IEF, SEC, and SDS-PAGE. The clarified concentrated UF retentate was removed from the system and aseptically filtered. It was stored at 2-8° C.

Membrane Cleaning

A stringent cleaning regime was employed in order to assure high cycle to cycle membrane water flux recovery. Cleaning steps were designed to mimic standard membrane cleaning in the dairy industry taking into consideration aspects of biopharmaceutical practices. The water flush steps were optimized to minimize water use while flushing out residual chemical for proper pH and conductivity values. The following cleaning cycles were carried out after every processing step provided in Tables 1 and 2 below:

TABLE 1
Ceramic membrane cleaning steps:

| | Step | Concentration | Volume | Time | Temp | pH |
|---|---|---|---|---|---|---|
| 1) | Water Flush | — | 16-20 L | 5 min. | 60° C. | 7.0 |
| 2) | NaOH Wash | 0.5 M | 1 | 10 min. | 60 | >11.5 |
| | Sodium Hypochlorite | 400 ppm | | | | |
| 4) | NaOH Wash | 0.5 M | 1 | 30 min. | 60 | >11.5 |
| | Sodium Hypochlorite | 400 ppm | | | | |
| 5) | Water Flush | — | 20-25 | 5 min. | 60 | 7.0 |
| 6) | Citric Acid Wash | 0.4 M | 1 | 30 min. | 60 | <2.75 |
| 7) | Water Flush | — | 16 | 10 min. | 60 | 7.0 |
| 8) | Sodium Hypochlorite | 300 ppm | 1 | 15 min. | 60 | >9.5 |
| | NaOH | 0.05 M | | | | |
| 9) | Water Flush | — | 12 | 10 min. | 60 | 7.0 |
| 10) | NaOH Storage | 0.1 M | 1 | | 20 | 10-12 |

TABLE 2
30 kDa PES membrane cleaning steps:

| | Step | Concentration | Volume | Time | Temp. | pH |
|---|---|---|---|---|---|---|
| 1) | USP Water Flush | — | 2 L/sqft | | 35° C. | 5.0 |
| 2) | NaOH Flush | 0.5 M | 2 L/sft | | 35 | >11.5 |
| | Sodium Hypochlorite | 250 ppm | | | | |
| 4) | NaOH Wash | 0.5 M | 2 L/sqft | 60 min. | 35 | >11.5 |
| | Sodium Hypochlorite | 250 ppm | | | | |
| 5) | USP Water Flush | — | 4 L/sqft | | 35 | 7.0 |
| 6) | Citric Acid Wash | 0.4 M | 2 L/sqft | 60 min | 35 | <2.75 |
| 7) | USP Water Flush | — | 4 L/sqft | | 35 | 7.0 |
| 10) | NaOH Storage | 0.1 M | | | 35 | 10-12 |

Prior to using a membrane for the first time, a normalized water permeability curve was made relating transmembrane pressure, temperature and water flux. Prior to use in an experiment, the normalized water permeability was checked to maintain a minimum 80% recovery of water flux. The ceramic membranes maintained a 95-105% recovery during development and the 30 kDa PES membranes maintained 80-90% recovery.

Aseptic Filtration

As seen in Graph J below, Pall Gelman Inc., makes a sterile filter made of Supor membrane with 0.8 um prefilter membranes and 0.2 um filter membranes combined in a cartridge. These cartridges contain 200 cm², the smallest membrane area available in capsule format for sterile filtration. An experiment was done to determine the filtration capacity of each capsule. Non-transgenic milk was clarified using dual TFF to produce a large quantity of clarified milk that would mimic the feed stream during aseptic processing. A 37 mm disk of Supor membrane was installed in a stainless steal normal flow holder and assembled with a digital pressure transducer and peristaltic pump. USP water was flushed through the entire system to wet the membrane and check for leaks. Clarified non-transgenic milk was then pumped through the system at a constant flow rate, and the pressure was recorded periodically. The data was fit to a line, which related throughput to pressure in the following graph. At 30 psig, the membrane would be plugged therefore throughput was extrapolated to 30 psig to determine capacity. The extrapolated capacity was 7343 ml for a 37 mm disk, which computes to 131 L for a 200 cm²capsule.

[image]

Membrane Storage

Once the cleaning protocol for the membranes was determined, storage conditions were tested. The membranes were stored in water or in 0.1 N sodium hydroxide after cleaning for 48 hours. The storage solution was rinsed out and the NWP was tested. The NWP was compared to the NWP after cleaning. The two NWP values post storage consistently within 10% of the NWP before storage. Since 0.1 N sodium hydroxide is anti-bacterial and anti-fungicidal, and the NWP did not decrease during storage, it was chosen as the storage agent.

Concentration vs. Product Flux

The liquid flux began to decrease almost immediately upon beginning the concentration and it continued to decrease steadily during the experiment. The last few points show a sharp drop in flux due to membrane fouling. In order to maintain optimum liquid flux during processing while operating at a low enough volume to allow for reasonable diafiltration time, a 1.5× to 3× milk concentration is recommended.

[image]

Tg Milk

IgG quantitation by protein A HPLC showed that both IgG1 antibody and liquid flux steadily declined with milk concentration. From the graph L below, 1.5 to 2.5× is reasonable for operating the dual TFF. SDS-PAGE showed no aggregation or degradation due to milk concentration.

[image]

Temperature vs. Product Flux

The IgG1 antibody mass flux through the microfiltration membrane reached a maximum at 27° C., at 20.3 gm/m2/hr, which is evident in the graph below. The optimum range of operation was 26° C.-29° C. Referring to Graph M below, IEF showed no modification of IgG1 antibody isoforms due to processing. SDS-PAGE was uninformative for the milk samples, and the clarified milk samples showed degradation bands. These degradation bands are present in initial milk samples from D035 and are lighter in the TFF clarified bulk material.

[image]

Flow Velocity and TMP vs. Product Flux

Each TMP gave an optimum flow velocity, but at 15 psi of TMP and 42 cm/s (14 lpm) of flow velocity, the IgG1 antibody flux was highest overall. The graph below shows a curve representative of the effects of flow velocity at each transmembrane pressure. IEF showed no change in isoforms due to processing, and SDS-PAGE showed similar results to the previous experiment.

[image]

As seen in Graph N, the first process tests showed a total recovery of 81% of IgG1 antibody from the milk pool. However, about 20% of it was aggregated.

The IEF bands looked the same at the end of the clarification as in the initial milk pool.

Also, samples from the middle diafiltration volumes showed very low concentrations of IgG1 antibody indicating samples were taken from unmixed areas of the UF feed reservoir. The experiment was repeated.

The second process test showed a 90% recovery of the IgG1 antibody, only 5%±0.5% was aggregated. The IEF gel showed no isoform modifications due to processing. SDS-PAGE showed slight aggregation and degradation bands, but these bands did not amount to significant percentages of aggregate or degraded protein since the final sample was 96.2% monomer, determined by size exclusion chromatography.

Due to low starting concentrations of IgG1 antibody, the protein A assay for IgG quantitation made determining the number of diafiltrations to recover IgG1 antibody difficult. Six diafiltrations gave 90% recovery, however five diafiltrations gave 170% recovery by protein A. Therefore, five to six difiltrations will probably be sufficient to recover IgG1 antibody.

After a number of engineering runs on the equipment used in the pilot plant to clarify milk, it was determined the equipment and procedures used required modification in order to produce clear clarified milk consistently. The equipment was removed from the GMP environment of the pilot plant to the development laboratory for extensive testing. The modifications made to the system included reducing the permeate piping and changing the location of the valves in the system to facilitate easier rinsing during the cleaning and sanitization steps. The cleaning protocols were slightly modified to improve the cleaning efficiency and reduce water usage. Process temperature ranges were determined. Finally, the process parameters were better defined in the GMP documentation.

The original design for the pilot equipment was constructed entirely of stainless steel. This design was cumbersome to clean since many long lengths of pipe needed to be disassembled from the process mode into the cleaning mode. Because of the length and inner diameter of the UF permeate piping, it was not effectively cleaned or rinsed during the cleaning protocol. A number of pieces were added to the MF system to facilitate cleaning, however their construction caused dead spaces for debris to accumulate. These problems were remedied by replacing the long UF permeate piping with ¼″ inner diameter tubing. The cleaning set-up was altered such that the top port of the MF membrane would be used for cleaning the permeate side of the membrane eliminating the need for the other pieces. The UF permeate tubing then remains on the UF during cleaning. Also, a large heat exchanger had been installed on the MF portion of the system, which allowed fine temperature control on the MF, but prevented controlling the UF temperature within the proper range for processing. The heat exchanger was removed from the system, and the chiller setting was adjusted to properly cool both systems within the proper temperature range. The final design is below. Equipment assembled for storage, sanitizing and processing. Configuration of equipment in an a preferred embodiment of the invention is provided in Graphs O and P below.

[image]

There were other simple modifications made to the equipment. After the UF system was tested using water to determine the cause of high pressure preventing adequate cross-flow across the UF membrane, it was determined the membranes were torqued down too hard, and the appropriate torque was 60 ft-pounds. The rotors and seals of the pump were also shedding. An 80-mesh screened gasket was inserted into the piping upstream of the UF membrane to catch large pieces and prevent flow restrictions and pressure build-up on the UF membrane. The UF retentate valve was moved such that it was adjacent to the UF reservoir. The spool piece that connected the MF permeate valve to the UF reservoir during processing was removed and the valve was connected directly to the reservoir. These modifications facilitate easier cleaning and rinsing during the cleaning steps, and also allow the entire system to be connected in process mode during sanitization and subsequent rinsing and clean water permeability testing.

Processing Changes

The TFF operation SOP and batch record for processing milk containing IgG1 antibody were modified to include ranges for cross-flow rates, transmembrane pressures, and temperatures for both the MF and UF systems. The temperature ranges were determined by a series of experiments. The parameters investigated are outlined in the table 3 below with the quality of the clarified milk produced. HEX refers to the use of a heat exchanger on the MF. A graph comparing the temperature ranges of the last three runs (5-7) is in Appendix B. 1.

TABLE 3
Processing Changes.

| | | | MF Temp | UF Temp | Chiller | |
|---|---|---|---|---|---|---|
| | Notebook | HEX | Range | Range | SP | |
| Run | Pages | (y/n) | (° C.) | (° C.) | (° C.) | Quality |
| 1 | 137-151 | Y | 22 | 30 | 22 | Cloudy |
| 2 | 152-156 | N | 25-29 | 22-27 | 20, 15 | Clear |
| 3 | 157-160, 173 | N | 25-30 | 24-28 | 15 | Clear |
| 4 | 162-162 | N | 20-30 | 23-29 | 15 | Cloudy |
| 5 | 169-172 | N | 20-26 | 21-24 | 10 | Clear |
| 6 | 176-179 | N | 20-26 | 21-24 | 10 | Clear |
| 7 | N/A | N | 20-27 | 21-24 | 10 | Clear |

All of the engineering runs on the pilot equipment produced either hazy or cloudy clarified milk. Operating at temperatures too high causes the clarified milk to look hazy and almost green in color, as opposed to clear and yellow. High temperature processing may cause various molecules in the milk to pass through the MF membrane that normally are retained, and it may affect the IgG1 antibody stability. When the process is run at the proper cross flow rates and transmembrane pressures, the pumps do not cause the temperature to increase out of control as was seen during the engineering runs. Haziness in the clarified milk was also caused by chemical residue from improper flushing during cleaning in some runs, and was determined by the pH of 9 in the sample (normally pH 6.7). By modifying the equipment and the cleaning procedure, the chemicals were adequately flushed from the system, as was shown by measuring the pH and conductivity of the rinse water from all streams.

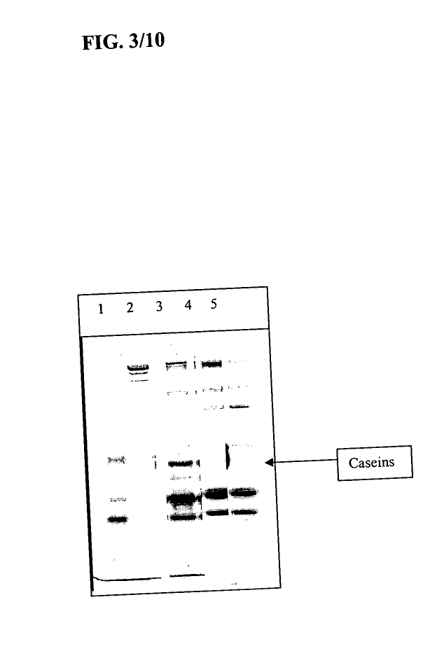

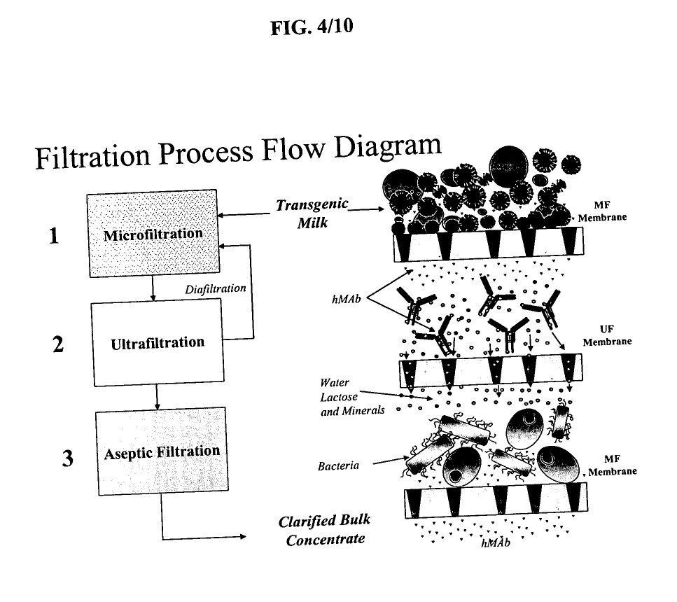

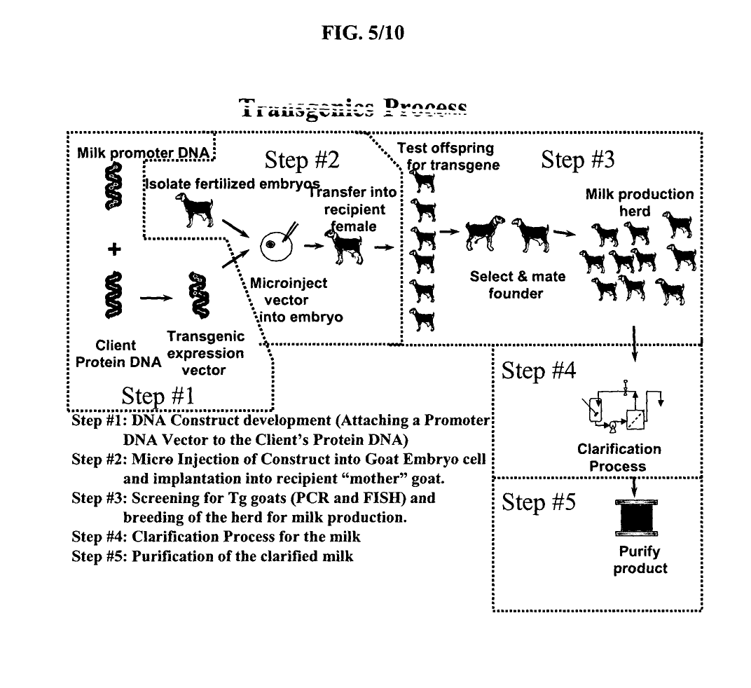

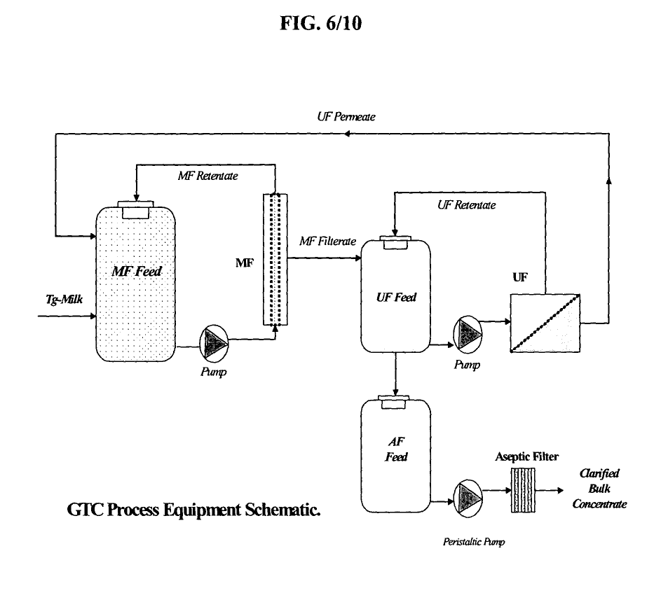

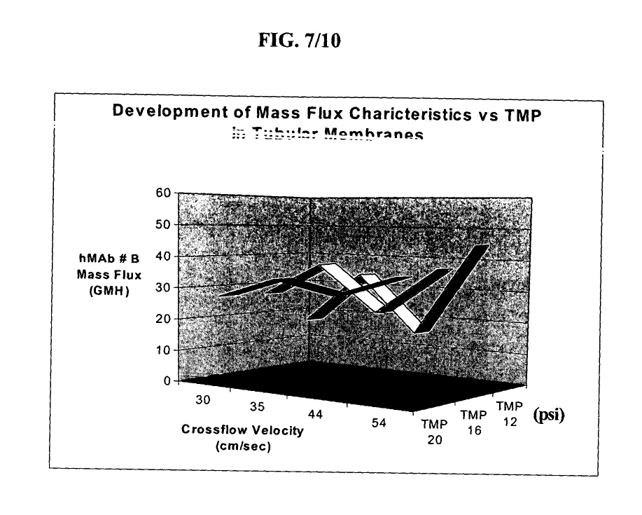

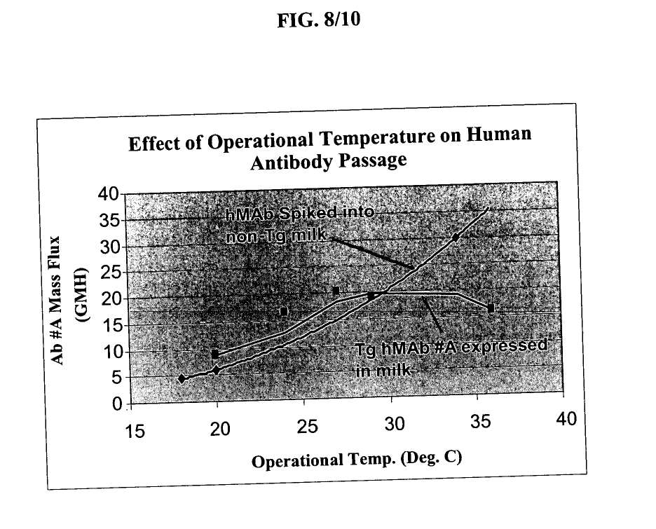

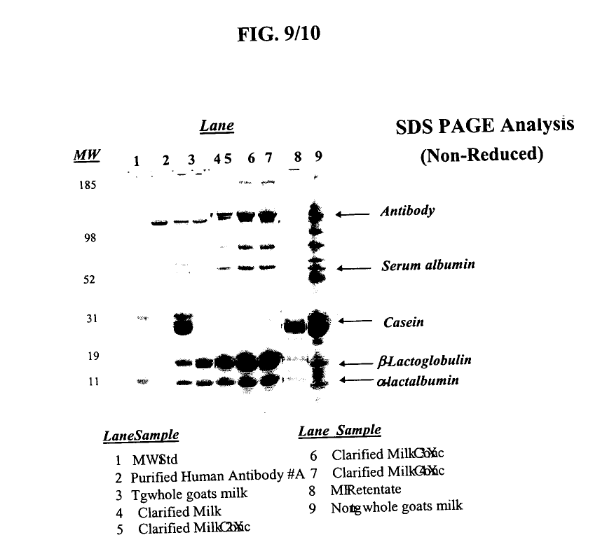

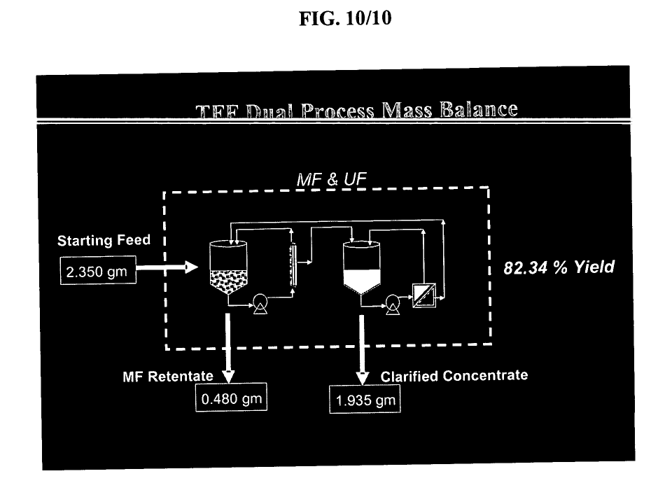

Operating at temperatures too low makes the clarified milk look cloudy with a whitish flocculent evenly dispersed throughout. When a heat exchanger was installed in the MF system, the temperature was easily controlled, but the clarified milk remained cloudy. According to Dairy and Biochemistry by P. F. Fox and P. L. H. McSweeny (1998) caseins are insoluble at their isoelectric points, and the insolubility range increases with increasing temperature. This suggests that more casein is removed by the MF at higher temperatures than at lower temperatures, and that process the MF at a lower temperature than the UF causes the soluble caseins that passed through the MF to become insoluble in the warmer UF. SDS-PAGE confirmed the phenomenon showing excess casein in the lower temperature run in comparison to clarified milk made during a bench scale run and a successful pilot scale run (below). Therefore, a balance was found between maintaining a high level of casein insolubility at the lowest possible temperature. According to the runs performed, running the MF at 22° C. was too low, while running it at 30° C. was too high. Maintaining the temperature near 25° C. for the majority of the run in the MF produced clear clarified milk reproducibly. SDS-PAGE gel comparisons are provided in FIG. 3. Referring to FIG. 3, Lane 1 shows the molecular weight standard. Lane 2 is cell culture IgG1 antibody. Lane 3 is the final clarified bulk from the engineering run on Apr. 17, 2001. Lane 4 is the final clarified bulk from pilot run 6 (proper temperature), and lane 5 is bench TFF clarified bulk material. The engineering run sample shows much more casein relative to the samples from the pilot run 6 and the bench clarified material.

Cleaning and Sanitization Changes

The equipment changes performed necessitated altering the cleaning and sanitization protocols. The cleaning protocol was run after every run in the table above. The retentate valve on the MF needed to be left half-open to facilitate proper rinsing during each rinse step since there is a long dead leg between the valve and the reservoir. After run 4, the cleaning protocol was run and the water consumption was tracked (Notebook 10586). The water used in this experiment was verified after runs 5, 6, and 7, and was recommended for use in GMP processing. As was stated before, the equipment alterations also allow the system to be sanitized in process mode. This was tested. The USP water required to rinse the sanitant from the system was also determined.

Operation

The actual steps taken to perform milk processing using dual TFF are described in the following sections. These include the entire process from sanitizing the systems, to processing, to cleaning, and to storing. The procedures were used on the equipment in the development lab during runs 5-7 and produced clear clarified milk.

Sanitization

To perform dual TFF using a ceramic 0.2 um microfiltration membrane and a 30 kda ultrafiltration membrane to clarify and concentrate transgenic goat milk from goat D035, the system must be sanitized with 0.1M sodium hydroxide. The equipment is assembled for sanitization and processing as above. 2L of 0.1M sodium hydroxide made with USP water is pumped through each system, with 15 LPM of cross flow on the MF and 1 LPM of cross flow on the UF. No retentate pressure is added to the MF, while 5 psi of pressure is added to the retentate of the UF. The permeate valves are completely open allowing the sodium hydroxide to recirculate around the entire system. The recirculation is done for 15 minutes, and then the solution is drained from the system through the bleed valves between the tanks and the pumps. USP water is used to rinse out the system by filling the tanks up completely with USP water whenever necessary. 1L of water is drained from each bleed valve. The retentate valves on the MF are half closed, and the permeate valve is directed completely to waste. The retentate and permeate valves on the UF are directed completely to waste. 12L of USP water is flushed through the MF retentate with a cross flow rate of 20 LPM. 4L of USP water is flushed through the MF permeate with a cross flow rate of 15-20 LPM and 6-8 psi of TMP. 7L of USP water is flushed through the UF retentate and permeate lines with a cross flow rate of 1 LPM, then the permeate is flushed with an additional 3L.

Using USP water (adding more if necessary), pump the MF at 20 LPM, increase the retentate pressure until the TMP of 15 psi is reached with no permeate pressure, then adjust the cross flow rate with pump speed to 15 LPM. Record the temperature (must be between 25-28° C.), pressures, and cross flow rate. Measure the permeate flow rate through the permeate drain valve. Repeat on the UF using 1 LPM of cross flow, and 5 psig of retentate pressure, and no permeate pressure (TMP of approximately 10 psig). Compare the permeate flow rates to those of the membranes' virgin water permeability. If the permeation rate is less than 80% of the original value, either re-clean the membranes or replace them.

Milk Processing

The milk must be pooled and raised to 15-20 C. The milk is pooled in the MF reservoir, then the MF permeate valve is closed, the retentate valve is opened, and the pump is turned on for a cross flow of 20 LPM. After 5 minutes the initial milk sample(s) are taken. The pressure is then increased for a TMP of 15 psig and cross flow rate of 15 LPM. The recirculation continues until the milk temperature reaches 20° C. Then the chiller is turned on at 10° C. and the MF permeate valve is opened to allow the milk to be concentrated to half of it's original volume on the microfiltration system by collecting the permeate of the ceramic membrane. The MF is run at 15 lpm cross flow rate with 15 psi of transmembrane pressure. The temperature of the MF should increase to and remain at 26° C.±2.0. The ultrafiltration system must then be started up at 0.8-1 LPM/sqft cross flow rate. The permeate flow rates of each membrane are measured through the permeate valves. The retentate and permeate pressures of the UF must be adjusted to cause the permeate flow rate to match the permeate flow rate of the MF. Once the UF permeate flow rate matches that of the MF. The systems should be run coupled for 5-6 diafiltration volumes. Once diafiltration is complete, the systems are disconnected, the MF is shut of, drained and cleaned, and the UF permeate is directed to drain until the volume of bulk clarified concentrate in the feed reservoir of the UF is concentrated to half it's volume for a total concentration of 4×. The UF is then drained, the bulk clarified concentrate is aseptically filtered, and the UF is cleaned.

Cleaning and Storing Protocols

The systems are disconnected according to the diagrams on page 14 of this report. The MF is rinsed with 20 L hot soft water (45-65° C.) with the retentate valves half open, and the permeate directed to drain. The valves are directed to recirculate solution back to the feed reservoir, and 2 L of hot 0.5 M sodium hydroxide with 400 ppm sodium hypochlorite is re-circulated for 5 minutes. The solution is drained from the system and replaced with 2 L of the same chemicals. The fresh solution is re-circulated for 30 minutes, then drained through the bleed valve. The system is flushed with 20 L of hot soft water through the half opened retentate valves. 4 L is flushed through the permeate only by recirculating the water on the retentate side of the membrane at 20 lpm with 6-8 psi of TMP. Remaining water is drained through the bleed valve. 2 L of hot 0.5 M citric acid is re-circulated through the system for 30 min at 20 LPM with 6-8 psi of TMP. The citric acid is then drained out through the bleed valve. 15 L of soft water is used to rinse out the retentate side of the MF, and 4 L is used to rinse out the permeate side as was done after the caustic step. 2 L of hot 0.05 M sodium hydroxide with 400 pm bleach was then re-circulated through the MF for 15 minutes and drained and rinsed out with 10 L of water on the retentate side and 4 L through the permeate as was done after the caustic step. The UF retentate and permeate lines are directed to drain for the initial water flush by directing the retentate valve to drain, and directing the entire permeate line to drain (not by the valve). Always run the pump at 1 LPM, i.e. if the retentate pressure is increased, the pump speed must also be increased to maintain 1 LPM. Rinse 4 L of USP water through both lines. Flush 2 L of 0.5 M sodium hydroxide with 250 ppm sodium hypochlorite made with USP water through both lines. Recirculate 2 L of fresh solution through the system with the permeate line attached to the feed reservoir, and the retentate valve open to the reservoir for 60 minutes. Drain the solution through the bleed valve. Direct both lines to drain as in the initial flush. Fill the reservoir with USP water and drain 1 L through the bleed valve. Flush 8 L through both lines, and an additional 4 L through the permeate line with 5 psi of retentate pressure. 2 L of 0.4 M citric acid are then re-circulated through the system for 60 minutes. The acid solution is drained through the bleed valve, then the reservoir is filled with USP water and 1 L is drained through the bleed valve. 8 L of water is flushed through both the retentate and permeate lines, then and additional 8 L is flushed through the permeate at a cross flow of 1 LPM across the membrane with 5 psi of retentate pressure. When both systems are cleaned and rinsed, they are assembled for storage (diagram above). 2 L of 0.1 M sodium hydroxide is poured into each feed vessel and pumped through the systems with the retentate and permeate valves open for recirculation, closed to waste, for 2 minutes. The vessels are then covered and status labeled as clean and stored in 0.1 M sodium hydroxide.

Process parameters have shown to be important in producing consistent material. The membranes used for the clarification are the CerCor ceramic 0.2 um pore size membrane, 1.5 sqft and the 30 kDa NMWCO Pall Filtron PES cassettes, 2 sq. ft. (2 cassettes). The temperature of the microfiltration system should be held between 26-29 C for optimum IgG1 antibody clarity and flux. The microfiltration system should be run at a retentate flow rate of 14 LPM (42 cm/s) with a transmembrane pressure of 15 psig. The milk should be concentration down to 40-70% of the volume of the original pool (1.5-2.5×). The ultrafiltration portion of the system should be run at 1.6-2 LPM retentate flow rate with 20-30 psig of feed pressure. Permeate flow rate should be matched to that of the microfiltration system by adjusting the permeate pressures. The final bulk clarified concentrate should be one-quarter the volume of the original milk pool (4× concentration).

Recombinant Production

A growing number of recombinant proteins are being developed for therapeutic and diagnostic applications. However, many of these proteins may be difficult or expensive to produce in a functional form and/or in the required quantities using conventional methods. Conventional methods involve inserting the gene responsible for the production of a particular protein into host cells such as bacteria, yeast, or mammalian cells, e.g., COS or CHO cells, and then growing the cells in culture media. The cultured cells then synthesize the desired protein. Traditional bacteria or yeast systems may be unable to produce many complex proteins in a functional form. While mammalian cells can reproduce complex proteins, they are generally difficult and expensive to grow, and often produce only mg/L quantities of protein. In addition, non-secreted proteins are relatively difficult to purify from procaryotic or mammalian cells as they are not secreted into the culture medium.

In general, the transgenic technology features, a method of making and secreting a protein which is not normally secreted (a non-secreted protein). The method includes expressing the protein from a nucleic acid construct which includes:

(a) a promoter, e.g., a mammary epithelial specific promoter, e.g., a milk protein promoter;

(b) a signal sequence which can direct the secretion of a protein, e.g. a signal sequence from a milk specific protein;

(c) optionally, a sequence which encodes a sufficient portion of the amino terminal coding region of a secreted protein, e.g., a protein secreted into milk, to allow secretion, e.g., in the milk of a transgenic mammal, of the non-secreted protein; and

(d) a sequence which encodes a non-secreted protein,

wherein elements (a), (b), optionally (c), and (d) are preferably operatively linked in the order recited.

In preferred embodiments: elements a, b, c (if present), and d are from the same gene; the elements a, b, c (if present), and d are from two or more genes.

In preferred embodiments the secretion is into the milk of a transgenic mammal.

In preferred embodiments: the signal sequence is the β-casein signal sequence; the promoter is the β-casein promoter sequence.

In preferred embodiments the non-secreted protein-coding sequence: is of human origin; codes for a truncated, nuclear, or a cytoplasmic polypeptide; codes for human serum albumin or other desired protein of interest.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Milk Specific Promoters

The transcriptional promoters useful in practicing the present invention are those promoters that are preferentially activated in mammary epithelial cells, including promoters that control the genes encoding milk proteins such as caseins, beta lactoglobulin (Clark et al., (1989) Bio/Technology 7: 487-492), whey acid protein (Gorton et al. (1987) Bio/Technology 5: 1183-1187), and lactalbumin (Soulier et al., (1992) FEBS Letts. 297: 13). Casein promoters may be derived from the alpha, beta, gamma or kappa casein genes of any mammalian species; a preferred promoter is derived from the goat beta casein gene (DiTullio, (1992) Bio/Technology 10:74-77). The milk-specific protein promoter or the promoters that are specifically activated in mammary tissue may be derived from either cDNA or genomic sequences. Preferably, they are genomic in origin.

DNA sequence information is available for all of the mammary gland specific genes listed above, in at least one, and often several organisms. See, e.g., Richards et al., J. Biol. Chem. 256, 526-532 (1981) (α-lactalbumin rat); Campbell et al., Nucleic Acids Res. 12, 8685-8697 (1984) (rat WAP); Jones et al., J. Biol. Chem. 260, 7042-7050 (1985) (rat β-casein); Yu-Lee & Rosen, J. Biol. Chem. 258, 10794-10804 (1983) (rat γ-casein); Hall, Biochem. J. 242, 735-742 (1987) (α-lactalbumin human); Stewart, Nucleic Acids Res. 12, 389 (1984) (bovine αs1 and κ casein cDNAs); Gorodetsky et al., Gene 66, 87-96 (1988) (bovine β casein); Alexander et al., Eur. J. Biochem. 178, 395-401 (1988) (bovine κ casein); Brignon et al., FEBS Lett. 188, 48-55 (1977) (bovine αS2 casein); Jamieson et al., Gene 61, 85-90 (1987), Ivanov et al., Biol. Chem. Hoppe-Sevler 369. 425-429 (1988), Alexander et al., Nucleic Acids Res. 17, 6739 (1989) (bovine β lactoglobulin); Vilotte et al., Biochimie 69, 609-620 (1987) (bovine α-lactalbumin). The structure and function of the various milk protein genes are reviewed by Mercier & Vilotte, J. Dairy Sci. 76, 3079-3098 (1993) (incorporated by reference in its entirety for all purposes). To the extent that additional sequence data might be required, sequences flanking the regions already obtained could be readily cloned using the existing sequences as probes. Mammary-gland specific regulatory sequences from different organisms are likewise obtained by screening libraries from such organisms using known cognate nucleotide sequences, or antibodies to cognate proteins as probes.

Although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

It should also be noted that while albumin is crystallized with various compounds, ethanol and mineral salts including phosphates industrial methods for crystallization with phosphates are not found in the literature. Through the preferred embodiments of the current invention it has now been found that human albumin can be crystallized advantageously with phosphate salts by utilizing in full extent the invented key process parameters and/or conditions of the current invention. The invented parameters and some variations thereof are listed and described above.

Accordingly, it is to be understood that the embodiments of the invention herein providing for an improved method of tangential flow filtration to generate a high yield of a molecule of interest from a given feedstream are merely illustrative of the application of the principles of the invention. It will be evident from the foregoing description that changes in the form, methods of use, and applications of the elements of the disclosed may be resorted to without departing from the spirit of the invention, or the scope of the appended claims.

### Prior Art Citations Incorporated by Reference

1. Andersson, 1966, “The Heterogeneity of Bovine Serum Albumin,” BIOCHIM. BIOPHYS. ACTA. 117:115-133.

2. Carter D C, et al., Crystals Of Serum Albumin For Use In Genetic Engineering And Rational Drug Design, U.S. Pat. No. 5,585,466.

3. Alun J, Morgan W, and Pickup R W (1993), Activity Of Microbial Peptidases, Oxidases, And Esterases In Lake Waters Of Varying Trophic Status, CAN J MICROBIOL 39(8):795-803.

4. Aranha-Creado H, and Fennington G J Jr (1997, Cumulative Viral Titer Reduction Demonstrated By Sequential Challenge Of A Tangential Flow Membrane Filtration System And A Direct Flow Pleated Filter Cartridge, PDA J PHARM SCI TECHNOL 51(5):208-212.

5. Aravindan G R, et al., (1997), Identification, Isolation, And Characterization Of A 41-Kilodalton Protein From Rat Germ Cell-Conditioned Medium Exhibiting Concentration-Dependent Dual Biological Activities, ENDOCRINOLOGY 138(8):3259-68.

6. Federspiel G, et al., (1991), Hybridoma Antibody Production In Vitro In Type II Serum-Free Medium Using Nutridoma-SP Supplements. Comparisons With In Vivo Methods, J IMMUNOL METHODS 145(1-2):213-221.

7. Kahn D W, et al., (2000), Purification Of Plasmid DNA By Tangential Flow Filtration, BIOTECHNOL BIOENG. 69(1): 101-106.

8. Kawahara H, et al., (1994), High-Density Culture Of FM-3A Cells Using A Bioreactor With An External Tangential-Flow Filtration Device, CYTOTECHNOLOGY 14(1):61-66.

9. Prado S M, et al., (1999), Development And Validation Study For The Chromatographic Purification Process For Tetanus Anatoxin On Sephacryl S-200 High Resolution, BOLL CHIM FARM. 138(7):364-368.

10. Ronco C, et al., (1994), On-Line Filtration Of Dialysate: Structural And Functional Features Of An Asymmetric Polysulfone Hollow Fiber Ultrafilter, INT J ARTIF ORGANS 17(10):515-520.

11. Strauss P R (1995), Use Of Filtron Mini-Ultrasettetm Tangential Flow Device And Filtron Microseptm Centrifugal Concentrators In The Early Stages Of Purification Of DNA Polymerases, BIOTECHNIQUES 18(1):158-160.

12. Porter, ed., HANDBOOK OF INDUSTRIAL MEMBRANE TECHNOLOGY, (Noyes Publications, Park Ridge, N.J., 1998) pp. 160-176.

13. Gabler et al., (1987), Principles of Tangential Flow Filtration: Applications to Biological Processing, in FILTRATION IN THE PHARMACEUTICAL INDUSTRY, pp. 453-490.

14. van Reis et al., U.S. Pat. No. 5,256,294; Tangential Flow Filtration Process And Apparatus.

15. Lenk, et al., U.S. Pat. No. 5,948,441; Method For Size Separation Of Particles.

16. van Reis et al., U.S. Pat. No. 5,490,937; Tangential Flow Filtration Process And Apparatus.

17. Marinaccio, et al., U.S. Pat. No. 4,888,115; Cross-Flow Filtration

What is claimed is:

1. A method for separating a molecular species of interest from a feedstream, comprising:
(a) filtering said feedstream by a tangential-flow filtration process through a filtration membrane having a pore size that separates said molecular species of interest from said feedstream, while maintaining flux at a level ranging from about 5 to 100% of transition point flux in the pressure-dependent region of the flux versus TMP curve, wherein transmembrane pressure is held substantially constant along the membrane at a level no greater than the transmembrane pressure at the transition point of the filtration, whereby said molecular species of interest is selectively separated from said feedstream such that said molecular species of interest retains its biological activity;
(b) filtering said feedstream by a microfiltration process; and
wherein said molecular species of interest is a protein.

2. The method of claim 1, further comprising fractionating said feedstream.

3. The method of claim 1, further comprising clarifying said feedstream.

4. The method of claim 1, further comprising diafiltering said feedstream.

5. The method of claim 1, further comprising concentrating said feedstream.

6. The method of claim 1, wherein the species of interest has a molecular weight of about 1 to 1000 kDa.

7. The method of claim 1, wherein all filtration stages are ultrafiltrations.

8. The method of claim 1, wherein said feedstream is milk.

9. The method of claim 1, wherein said feedstream is a cell lysate solution.

10. The method of claim 1, wherein said protein is a biopharmaceutical.

11. The method of claim 8, wherein the condition of said milk is selected from one of the following states:
a) raw;
b) diluted;
c) treated with a buffer solution;
d) chemically treated; and
e) partially evaporated.

12. The method of claim 2, wherein said fractionation step utilizes ceramic filtration membranes.

13. The method of claim 3, wherein said clarification step utilizes ceramic filtration membranes.

14. The method of claim 2, wherein said fractionation step utilizes polymeric filtration membranes.

15. The method of claim 3, wherein said clarification step utilizes polymeric filtration membranes.

16. The method of claim 2, wherein said fractionation step utilizes cellulose filtration membranes.

17. The method of claim 3, wherein said clarification step utilizes cellulose filtration membranes.

18. The method of claim 2, further comprising optimizing systematic parameters.

19. The method of claim 18, wherein said systematic parameters include temperature, feedstream flow velocity, transmembrane pressure, feedstream concentration and diafiltration volume.

20. The method of claim 3, further comprising optimizing systematic parameters.

21. The method of claim 20, wherein said systematic parameters include temperature, feedstream flow velocity, transmembrane pressure, feedstream concentration and diafiltration volume.

22. The method of claim 1 wherein said molecular species of interest are biological entities selected from the group consisting of proteins, immunoglobulins, polypeptides, peptides, glycoproteins, RNA and DNA.

23. The method of claim 19, wherein the optimal temperature range is from 15° C. to 50° C.

24. The method of claim 19, wherein the optimal temperature range is from 20° C. to 35° C.

25. The method of claim 19, wherein the optimal temperature range is from 25° C. to 29° C.

26. The method of claim 21, wherein the optimal temperature range is from 15° C. to 50° C.

27. The method of claim 21, wherein the optimal temperature range is from 20° C. to 35° C.

28. The method of claim 21, wherein the optimal temperature range is from 25° C. to 29° C.

29. The method of claim 19, wherein the feedstream flow velocity is from 10 cm/sec to 100 cm/sec.

30. The method of claim 19, wherein the feedstream flow velocity is from 20 cm/sec to 60 cm/sec.

31. The method of claim 19, wherein the feedstream flow velocity is from 25 cm/sec to 45 cm/sec.

32. The method of claim 21, wherein the feedstream flow velocity is from 10 cm/sec to 100 cm/sec.

33. The method of claim 21, wherein the feedstream flow velocity is from 20 cm/sec to 60 cm/sec.

34. The method of claim 21, wherein the feedstream flow velocity is from 25 cm/sec to 45 cm/sec.

35. The method of claim 19, wherein the transmembrane pressure ranges from 2 psi to 40 psi.

36. The method of claim 19, wherein the transmembrane pressure ranges from 5 psi to 30 psi.

37. The method of claim 19, wherein the transmembrane pressure ranges from 10 psi to 20 psi.

38. The method of claim 21, wherein the transmembrane pressure ranges from 2 psi to 40 psi.

39. The method of claim 21, wherein the transmembrane pressure ranges from 5 psi to 30 psi.

40. The method of claim 21, wherein the transmembrane pressure ranges from 10 psi to 20 psi.

41. The method of claim 19, wherein the feedstream concentration is from 0.25× to 4× natural milk.

42. The method of claim 19, wherein the feedstream concentration is from 0.5× to 3× natural milk.

43. The method of claim 19, wherein the feedstream concentration is from 1.0× to 2× natural milk.

44. The method of claim 21, wherein the feedstream concentration is from 0.25× to 4× natural milk.

45. The method of claim 21, wherein the feedstream concentration is from 0.5× to 3× natural milk.

46. The method of claim 21, wherein the feedstream concentration is from 1.0× to 2× natural milk.

47. The method of claim 19, wherein the diafiltration volume range is from 1× to 20× the volume of concentrated MF retentate.

48. The method of claim 19, wherein the diafiltration volume range is from 3× to 15× the volume of concentrated MF retentate.

49. The method of claim 19, wherein the diafiltration volume range is from 5× to 10× the volume of concentrated MF retentate.

50. The method of claim 21, wherein the diafiltration volume range is from 1× to 20× the volume of concentrated MF retentate.

51. The method of claim 21, wherein the diafiltration volume range is from 3× to 15× the volume of concentrated MF retentate.

52. The method of claim 21, wherein the diafiltration volume range is from 5× to 10× the volume of concentrated MF retentate.

53. The method of claim 2, wherein ultrafiltration membranes are used for all filtering steps.

54. The method of claim 5, wherein ultrafiltration membranes are used for all filtering steps.

55. The method of claim 8, wherein said milk is treated with a solution selected from the group consisting of:
a) water;
b) a buffered aqueous salt solution;
c) chelating agent;
d) acid solution; and
e) alkali solution.

56. The method of claim 4, wherein said diafiltration utilizes ultrafiltration permeate.

57. The method of claim 4, wherein said diafiltration utilizes water.

58. The method of claim 4, wherein said diafiltration utilizes a buffered salt solution.

59. The method of claim 1, wherein the membranes used are cleaned with solutions of a temperature greater than 20° C.

60. The method of claim 1, wherein the membranes used are cleaned with solutions ranging in temperature from 20° C. to 70° C.

61. The method of claim 1, wherein the membranes used are cleaned with solutions ranging in temperature from 40° C. to 60° C.

62. The method of claim 1, wherein the membranes used are cleaned with an acid solution.

63. The method of claim 1, wherein the membranes used are cleaned with an alkali solution.

64. The method of claim 1, wherein the membranes used are cleaned with a hypochlorite solution.

65. The method of claim 62, 63 or 64, further comprising a water rinse following the use of the selected solution.

66. The method of claim 1, wherein the membranes used are sanitized prior to use with a hydroxide solution.

67. The method of claim 1, wherein the membranes used are sanitized prior to use with an alcohol solution.

68. The method of claim 1, wherein the membranes used are sanitized prior to use with a hypochlorite solution.

69. The method of claim 1, wherein the membranes used are cleaned for a period of from 20 minutes to 45 minutes.

70. The method of claim 1, further comprising filtering the filtrate from the filtration in a second tangential-flow filtration stage through a membrane having a smaller pore size than the membrane used in the first filtration stage, and recycling the filtrate of this second filtration stages back to the first filtration stage, whereby the process is repeated.
